SEQUENCE LISTING (21 sequences)

SEQ ID NO: 1 - protein, 12 aa, Artificial Sequence
  C11 VL-CDR1
RASQSVSSSYLA

SEQ ID NO: 2 - protein, 7 aa, Artificial Sequence
  C11 VL-CDR2
GASSRAT

SEQ ID NO: 3 - protein, 9 aa, Artificial Sequence
  C11 VL-CDR3
QQAIGFPQT

SEQ ID NO: 4 - protein, 10 aa, Artificial Sequence
  C11 VH-CDR1
GFTFSSYAMS

SEQ ID NO: 5 - protein, 17 aa, Artificial Sequence
  C11 VH-CDR2
AISGSGGSTYYADSVKG

SEQ ID NO: 6 - protein, 7 aa, Artificial Sequence
  C11 VH-CDR3
TLAAFDY

SEQ ID NO: 7 - protein, 108 aa, Artificial Sequence
  C11-VL
EIVLTQSPGTLSLSPGERATLSCRASQSVSSSYLAWYQQKPGQAPRLLIYGASSRATGIPDRFSGSGSGTDFTLTISRLEPEDFAVYYCQQAIGFPQTFGQGTKVEIK

SEQ ID NO: 8 - protein, 116 aa, Artificial Sequence
  C11-VH
EVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSS

SEQ ID NO: 9 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 10 - protein, 15 aa, Artificial Sequence
  AKKAS linker
GGGAKGGGGKAGGGS

SEQ ID NO: 11 - protein, 15 aa, Artificial Sequence
  DDS linker
GGGGDGGGGDGGGGS

SEQ ID NO: 12 - protein, 15 aa, Artificial Sequence
  SAD linker
GSADGGSSAGGSDAG

SEQ ID NO: 13 - protein, 15 aa, Artificial Sequence
  SES linker
GGGGSGGGGEGGGGS

SEQ ID NO: 14 - protein, 15 aa, Artificial Sequence
  (G4S)3 linker
GGGGSGGGGSGGGGS

SEQ ID NO: 15 - protein, 201 aa, Artificial Sequence
  IGF1-C11-VH-AKKAS-variant
GPETLCGAELVDALQFVCGDRGFYFNKPTGYGSSSRRAPQTGIVDECCFRSCDLRRLEMYCAPLKPAKSAGGGAKGGGGKAGGGSEVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSS

SEQ ID NO: 16 - protein, 201 aa, Artificial Sequence
  IGF1-C11-VH-DDS-variant
GPETLCGAELVDALQFVCGDRGFYFNKPTGYGSSSRRAPQTGIVDECCFRSCDLRRLEMYCAPLKPAKSAGGGGDGGGGDGGGGSEVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSS

SEQ ID NO: 17 - protein, 201 aa, Artificial Sequence
  IGF1-C11-VH-SAD-variant
GPETLCGAELVDALQFVCGDRGFYFNKPTGYGSSSRRAPQTGIVDECCFRSCDLRRLEMYCAPLKPAKSAGSADGGSSAGGSDAGEVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSS

SEQ ID NO: 18 - protein, 201 aa, Artificial Sequence
  IGF1-C11-VH-SES-variant
GPETLCGAELVDALQFVCGDRGFYFNKPTGYGSSSRRAPQTGIVDECCFRSCDLRRLEMYCAPLKPAKSAGGGGSGGGGEGGGGSEVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSS

SEQ ID NO: 19 - protein, 201 aa, Artificial Sequence
  IGF-1-C11-VH-(G4S)3-variant
GPETLCGAELVDALQFVCGDRGFYFNKPTGYGSSSRRAPQTGIVDECCFRSCDLRRLEMYCAPLKPAKSAGGGGSGGGGSGGGGSEVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSS

SEQ ID NO: 20 - protein, 555 aa, Artificial Sequence
  IGF1-C11(scFV-Fc)-(G4S)3-variant
GPETLCGAELVDALQFVCGDRGFYFNKPTGYGSSSRRAPQTGIVDECCFRSCDLRRLEMYCAPLKPAKSAGGGGSGGGGSGGGGSEVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQAIGFPQTFGQGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 444 aa, Artificial Sequence
  IGF1-C11(SIP)-(G4S)3-variant
GPETLCGAELVDALQFVCGDRGFYFNKPTGYGSSSRRAPQTGIVDECCFRSCDLRRLEMYCAPLKPAKSAGGGGSGGGGSGGGGSEVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQAIGFPQTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

(19) United States
(12) Patent Application Publication
(10) Pub. No.: US 2020/0165326 A1
(43) Pub. Date: May 28, 2020

(54) IMMUNOCONJUGATES WITH OPTIMIZED LINKERS AND ORIENTATION

(71) Applicant: Philogen S.p.a., Siena (IT)

(72) Inventors: Sarah Wulhfard, Baden (CH); Mattia Matasci, Zurich (CH)

(21) Appl. No.: 16/487,991

(22) PCT Filed: Feb. 20, 2018
(86) PCT No.: PCT/EP2018/054161

§ 371 Date: Aug. 22, 2019

(30) Foreign Application Priority Data
Feb. 24, 2017 (GB) 1703022.2
Jun. 9, 2017 (GB) 1709206.5

(51) Int. Cl. C07K 16/18; C07K 14/65; A61K 9/00; A61K 39/395; A61P 19/02

(52) U.S. Cl. CPC C07K 16/18; C07K 14/65; A61K 9/0019; A61K 39/39591; A61K 38/00; C07K 2319/30; C07K 2317/626; C07K 2317/565; A61P 19/02

(57) ABSTRACT

The present invention relates to fusion proteins comprising a growth factor linked to the N-terminus of an antibody via a linker peptide, in particular, wherein said growth factor iS IGF-1 and said antibody is directed against collagen.

### SUMMARY OF THE INVENTION

The present invention relates to immunoconjugates with optimized linkers and orientation. These conjugates are fusion proteins comprising an antibody and a growth factor, conjugated by a given linker.

The present invention relates to immunoconjugates targeting epitopes present in various cartilage components, and being conjugated to growth factors, like insulin-like growth factor (IGF), or a fragment or a subunit thereof, wherein said antibody, or fragment or derivative thereof, and said growth factor, or fragment or subunit thereof, are covalently linked by a peptide linker and used for the regeneration of cartilage and other fibrous structures.

### INTRODUCTION AND BACKGROUND OF THE INVENTION

Recombinant IGF1 is a biopharmaceutical which is used for various clinical applications. It is administered to individuals with growth defects, such in dwarfism. In addition, IGF1 is frequently used by body-builders as intramuscular injection, in order to promote muscle growth. As IGF1, like many other growth factors, usually does not preferentially localize at sites of disease on its own, it has been understood that linking them to antibodies for delivery to diseased tissue can provide significant therapeutic advantage by improving potency and reducing side effects.

In order to improve the therapeutic index—i.e., the ratio between the amount of growth factor that causes the therapeutic effect to the amount that causes toxicity, IGF1 can be fused or conjugated to a suitable monoclonal antibody, antibody fragment, or antibody derivative, which then serves as a pharmacodelivery vehicle.

### Arthritis and Osteoarthritis

Arthritis represents one of the relevant targets for the development of new therapies. Arthritis affects approximately 80% of people over the age of 55 in the United States. Injury, a weakened immune system, and/or hereditary factors can trigger the onset of arthritis. There are hundreds of types of arthritis that share similar symptoms including inflammation, joint pain, and progressive deterioration of joint surfaces over time. The joints may lose normal contour, excessive amounts of fluid may build up inside the joint along with pieces of floating debris. Arthritis may affect the joints in the spine, which enable the body to bend and twist. Part of the problem may be the body's response to arthritis, which is to manufacture extra bone to stop joint movement. The extra bone is called a bone spur or bony overgrowth.

The most common forms of arthritis are rheumatoid arthritis, osteoarthritis, fibromyalgia, psoriatic arthritis, gout, lupus, juvenile arthritis and ankylosing spondylitis.

Sometimes called degenerative joint disease or degenerative arthritis, osteoarthritis (OA) is the most common chronic condition of the joints, affecting approximately millions of patients worldwide. OA can affect any joint, but it occurs most often in knees, hips, lower back and neck, small joints of the fingers and the bases of the thumb and big toe.

In normal joints, cartilage covers the end of each bone. Cartilage provides a smooth, gliding surface for joint motion and acts as a cushion between the bones. In OA, the cartilage breaks down, causing pain, swelling and problems moving the joint. As OA worsens over time, bones may break down and develop growths called spurs. Bits of bone or cartilage may chip off and float around in the joint. In the body, an inflammatory process occurs and cytokines (proteins) and enzymes develop that further damage the cartilage. In the final stages of OA, the cartilage wears away and bone rubs against bone leading to joint damage and more pain.

### Ligament or Tendon Injuries

Ligament or tendon injuries can occur as symptoms of increasing age, as well as due to chronic strain and acute injury. Currently, when a rupture takes place the only therapeutic option is surgery in which the torn ligament or tendon is generally replaced by a substitute graft. They constitute an important medical need. For instance reconstructive surgeries of Anterior Cruciate Ligament (ACL) in the USA only were estimated to be around 75,000 in 2009 [Lyman et al. (2009)]. The grafts used to replace the ACL include patellar tendon autograft (autograft comes from the patient), hamstring autograft or quadriceps autograft. However, unsatisfactory outcome of surgery due to rupture or stretching of the reconstructed ligament or poor surgical technique is possible [Freedman et al. (2003), Brown & Carson (1999)]. For this reason, any technique or pharmacological intervention that could improve the outcome of the surgery would be highly important.

Insulin-Like Growth Factor 1 as Active Component

The insulin-like growth factors (IGFs) are proteins with high sequence similarity to insulin. They are members of a family of insulin related peptides that include relaxin and several peptides isolated from lower invertebrates. In 1976, Rinderknecht and Humber isolated two active substances from human serum, which owing to their structural resemblance to proinsulin were renamed “insulin-like growth factor 1 and 2” (IGF-1 and IGF-2).

IGF-1 is a small peptide consisting of 70 amino acids with a molecular weight of 7649 Da. Similar to insulin, IGF-1 has an A and B chain connected by disulphide bonds. The C peptide region has 12 amino acids. The structural similarity to insulin explains the ability of IGF-1 to bind (with low affinity) to the insulin receptor.

The primary action of IGF-1 is mediated by binding to its specific receptor, the insulin-like growth factor 1 receptor (IGF1R), which is present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation.

Numerous clinical studies suggest that IGF-1 works to protect and repair cartilage tissue. Because this regenerative effect of IGF-1 is believed to offset the damage inflicted by reactive immune mediators, such as cytokines, to the cartilage, IGF-1 could be in theory regarded as a good candidate for the treatment of osteoarthritis.

However, IGF-1 has poor anabolic efficacy in cartilage in osteoarthritis (OA), partly because of its sequestration by abnormally high levels of extracellular IGF-binding proteins (IGFBPs) present in the serum and partly because of its short half-life.

One way to maximize the therapeutic activity of IGF-1 is to administer it in the joints affected by OA through intra-articular injections. An even better way to maximize the therapeutic activity of IGF-1 is to conjugate it to a protein capable of binding a target present in OA. The IGF1 fusion protein will then remain in the diseased tissue thus exerting its biological functions for a longer time. An even better way is to administer the IGF-1 fusion protein with intra-articular injections.

### Collagen as Antibody Target

Collagens are the major structural components of the extracellular matrix. A coordinated and regulated expression of the different collagens is important for correct development in vertebrates and collagen mutations are involved in several inherited connective tissue disorders. Among them, collagen type II (also called collagen II, or COL2A1) is the most abundant in cartilage [Strom and Upholt (1984), Cheah et al. (1985)]. COL2A1 is synthetized by chondrocytes during embryogenesis and de novo in pathological conditions in the adult. COL2A1 is a homotrimer composed of three a1(11) chains. These are secreted as long immature procollagen molecules that undergo proteolytic cleavage by collagenases in the extracellular environment, thereby forming the mature type II collagen. COL2A1 forms heteropolymers with collagen IX and collagen XI, creating the fibrillar network typical of cartilage [Eyre D. (2002)]. It has been known since the late 1980s that mutations in the COL2A1 gene are the cause of several hereditary disorders related to the abnormal development of bones and cartilage, including spondyloepiphyseal dysplasia congenital type [Lee B. et al. (1989)], spondyloepimetaphyseal dysplasia strudwick type and many others. Moreover different techniques have been used to investigate the expression of COL2A1 in normal and rheumatoid human articular cartilage.

Normal COL2A1 is expressed evenly in healthy tissue, while diseased joints show strong enhancement of type II collagen [Aigner et al. (1992)]. This evident change in the extracellular matrix composition is due to a failure of maintaining the homeostasis of the cartilage fibrillar network [Gouttenoire et al. (2004)]. COL2A1 is reasonably well conserved between mouse, rat and man.

However, in addition to COL2A1, various other components are present in human cartilage. An antibody capable of binding more cartilage components (i.e., not only to COL2A1, but also, e.g., collagen type, also called collagen I or COL1A1) may have superior properties in order to promote a more physiological regeneration of cartilage and other fibrous body structures.

### Antibodies Against Collagen and Fusion Proteins

Cartilage is a tissue that protects the ends of long bones at the joints and is a component of many body parts. Cartilage is composed of specialized cells called chondrocytes that produce an abundant extracellular matrix. The extracellular matrix is a complex of self-assembled macromolecules. It is composed predominantly of collagens, non-collagenous glycoproteins, hyaluronan and proteoglycans.

In principle collagens could be considered as a target for pharmacodelivery applications.

In WO2016/016269 the current applicants have disclosed an anti-collagen antibody named “C11” which has unique biological properties as it binds both collagen II and to collagen I. The C11 antibody displayed a good staining of vascular structures of various diseased tissues (e.g. SKRC-52 renal cell carcinoma, F9 murine teratocarcinoma, mouse paw from RA model). The C11 antibody has been studied in biodistribution and immuno-histochemistry (IHC) studies in a rat MMT model of OA, and in knee joint and synovium from human OA patients. In addition, the C11 antibody also binds to chondrocytes, to damaged cartilage and to the subchondral bone. The C11 antibody has therefore the potential to target therapeutics to osteoarthritic joints.

In contrast to the complex cartilage binding properties of C11, the applicants have also described in WO2016/016269 another anti-collagen antibody named “F9”, which specifically recognizes collagen II structures, but does not bind to collagen I.

WO2008/135734 described antibodies against oxidized collagen II, in particular the clone 1-11E which recognizes an epitope specifically contained in the oxidized form of collagen II. This antibody 1-11E binds only to damaged OA cartilage (pericellular staining of the extracellular matrix of cartilage tissues) but not to normal cartilage in immunochemistry. A 1-11E diabody was able to localize in the inflamed paw of an arthritis mouse model as well at the site of injury in a mouse OA model.

WO2008/135734 also disclosed antibodies conjugated to a cytokine or to a cytokine receptor. The production of fusion proteins such as 1-11E fused with IFN-beta or 1-11E fused with TNFR2-Fc.

The attachment of a growth factor to the antibody molecule can take place by means of a peptide linker [Chen et al. (2013)]. Besides the basic role in linking the functional domains together (as in flexible and rigid linkers) or releasing free functional domain in vivo (as in in-vivo cleavable linkers), linkers may offer many other advantages for the production of fusion proteins, such as improving biological activity, increasing expression yield, and achieving desirable pharmacokinetic profiles.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a,” “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to one aspect, the present invention relates to a fusion protein comprising an antibody or a fragment or derivative thereof retaining target binding properties, and a growth factor, or a fragment or a subunit thereof retaining growth factor activity, wherein said antibody, or fragment or derivative thereof, and said growth factor, or fragment or subunit thereof, are linked by a peptide linker, wherein the peptide linker is fused to the N-terminus of at least one peptide chain of the antibody, or the fragment or derivative thereof

This means that in this embodiment, the growth factor is fused to the part of the antibody that also comprised the antigen binding domains, namely the variable domains (see FIG. 9).

This means that the fusion protein has the following N->C orientation:

N-Growth factor-Linker-Antibody-C

This configuration is utterly different from other immunoconjugates, where the toxin, growth factor or cytokine is fused or conjugated to the constant domain (often to the C-Terminus thereof), to not interfere with target binding of the antibody, like, e.g., SS1P, which comprises an anti-mesothelin antibody Fv, the CH1 part of which is linked to the PE38 exotoxin.

The inventors have surprisingly shown that despite these considerations, the fusion of the growth factor-linker to the variable domain of the antibody does not interfere with the target binding of the latter.

In this context, it is important to understand that U.S. Pat. No. 8,394,378 discloses antibodies binding human collagen II, and suggests growth factors, cytokines and anti-inflammatory agents may be coupled thereto. In such conjugate, U.S. Pat. No. 8,394,378 suggests that the therapeutic protein may be directly linked to the C-terminus of the antibody of the invention via an amide bond or a peptide linker. Hence, U.S. Pat. No. 8,394,378 suggests the opposite arrangement compared to the arrangement as set forth above, clearly teaching away from the latter.

In one embodiment of the invention, the antibody, or fragment or derivative thereof, is specifically binding to a collagen. In one other embodiment of the invention, the antibody, or fragment or derivative thereof, is capable of binding both collagen I and collagen II. In one other embodiment of the invention, said collagen is human collagen, canine collagen or equine collagen.

As used herein, an “antibody”, also synonymously called “immunoglobulin” (Ig), is generally comprising four polypeptide chains, two heavy (H) chains and two light (L) chains, and is therefore a multimeric protein, or an equivalent Ig homologue thereof (e.g., a camelid nanobody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins; Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype.

An “antibody derivative or fragment”, as used herein, relates to a molecule comprising at least one polypeptide chain derived from an antibody that is not full length, including, but not limited to (i) a Fab fragment, which is a monovalent fragment consisting of the variable light (V_{L}), variable heavy (V_{H}), constant light (C_{L}) and constant heavy 1 (C_{H}1) domains; (ii) a F(ab′)2 fragment, which is a bivalent fragment comprising two Fab fragments linked by a disulfide bridge at the hinge region; (iii) a heavy chain portion of a F_{ab} (F_{d}) fragment, which consists of the V_{H} and C_{H}1 domains; (iv) a variable fragment (Fᵥ) fragment, which consists of the V_{L} and V_{H} domains of a single arm of an antibody, (v) a domain antibody (dAb) fragment, which comprises a single variable domain; (vi) an isolated complementarity determining region (CDR); (vii) a single chain F, Fragment (scFᵥ); (viii) a diabody (db), which is a bivalent, bispecific antibody in which V_{H} and V_{L} domains are expressed on a single polypeptide chain, but using a linker that is too short to allow for pairing between the two domains on the same chain, thereby forcing the domains to pair with the complementarity domains of another chain and creating two antigen binding sites; (ix) a linear antibody, which comprises a pair of tandem F, segments (V_{H}-C_{H}1-V_{H}-C_{H}1) which, together with complementarity light chain polypeptides, form a pair of antigen binding regions; (x) a SIP (Small Immuno Protein), which comprises two single chain antibodies (scFᵥ) crosslinked to one another by means of one or more CH domains (e.g., V_{H}-V_{L}-C_{H}4-C_{H}4-V_{L}-V_{H}) as described in WO2003/076469, (xi) scFv-FC, i.e., a construct where a scFv is fused, through an hinge region, to C_{H}2-C_{H}3 (or directly to a C_{H}1-C_{H}2-C_{H}3 region), i.e., lacking the CL domain, and (xii) other non-full length portions of immunoglobulin heavy and/or light chains, or mutants, variants, or derivatives thereof, alone or in any combination. In any case, said derivative or fragment retains target binding properties.

The VH and VL can be subdivided into regions of hyper-variability, termed complementarity determining regions (“CDRs”), interspersed with regions that are more conserved, termed framework regions (“FR”). Each VH and VL is composed of three CDRs and four FRs, arranged from amino-terminus to carboxy-terminus in the following order: FR1, CDR1, FR2, CDR2, FR3, CDR3, FR4. Herein, the three CDRs of the heavy chain are referred to as “VH-CDR1, VH-CDR2, and VH-CDR3” and the three CDRs of the light chain are referred to as “VL-CDR1, VL-CDR2 and VL-CDR3”.

In one embodiment of the present invention, the antibody is a monoclonal antibody selected from any of the group consisting of antibody.

a) hybridoma-derived antibody

b) chimerized antibody

c) humanized antibody, and/or

d) human antibody.

The hybridoma technique is well known in the art, and described by Köhler and Milstein [Köhler and Milstein (1975)]. Methods for the production and/or selection of chimeric, humanised and/or human mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies.

Quote generally, the antibody can be mammalized. This term refers to antibodies which comprise heavy and light chain variable region sequences from a mammal species (e.g., a mouse) but in which at least a portion of the VH and/or VL sequence has been altered to be more like “mammal of interest,” see for example, humanized, caninized, equinized or felinized antibodies defined herein. Such mammalized antibodies include, but are not limited to, bovanized, camelized, caninized, equinized, felinized antibodies, and their concept is similar to that of humanized antibodies. Antibody mammalization, including caninization and equinization, is disclosed, inter alia, in US 20160002324.

According to yet another embodiment, the antibody is a monoclonal antibody selected from any of the group consisting of

canine or caninized antibody, and /or

equine or equinized antibody

“Caninized” forms of non-canine (e.g., human or murine) antibodies are genetically engineered antibodies that contain minimal sequence derived from non-canine immunoglobulin. Caninized antibodies are canine immunoglobulin sequences (recipient antibody) in which hypervariable region residues of the recipient are replaced by hypervariable region residues from a non-canine species (donor antibody) such as man or mouse having the desired specificity, affinity, and capacity. In some instances, framework region (FR) residues of the canine immunoglobulin sequences are replaced by corresponding non-canine residues. Furthermore, caninized antibodies may include residues that are not found in the recipient antibody or in the donor antibody. These modifications are made to further refine antibody performance. In general, the caninized antibody will include substantially all of at least one, and typically two, variable domains, in which all or substantially all of the hypervariable regions correspond to those of a non-canine immunoglobulin sequence and all or substantially all of the FRs are those of a canine immunoglobulin sequence. The caninized antibody optionally also will comprise a complete, or at least a portion of an immunoglobulin constant region (Fc), typically that of a canine immunoglobulin sequence. In this embodiment, non canine CDRs are grafted onto canine frameworks.

“Equinized” forms of non-equine (e.g., human or murine) antibodies are genetically engineered antibodies that contain minimal sequence derived from non-equine immunoglobulin. Equinized antibodies are equine immunoglobulin sequences (recipient antibody) in which hypervariable region residues of the recipient are replaced by hypervariable region residues from a non-equine species (donor antibody) such as man or mouse having the desired specificity, affinity, and capacity. In some instances, framework region (FR) residues of the equine immunoglobulin sequences are replaced by corresponding non-equine residues. Furthermore, equinized antibodies may include residues that are not found in the recipient antibody or in the donor antibody. These modifications are made to further refine antibody performance. In general, the equinized antibody will include substantially all of at least one, and typically two, variable domains, in which all or substantially all of the hypervariable regions correspond to those of a non-equine immunoglobulin sequence and all or substantially all of the FRs are those of a equine immunoglobulin sequence. The equinized antibody optionally also will comprise a complete, or at least a portion of an immunoglobulin constant region (Fc), typically that of a equine immunoglobulin sequence. In this embodiment, non-equine CDRs are grafted onto equine frameworks.

Routine methods exist to create antibody libraries or peptide libraries, to then select, from such libraries, suitable binders against almost all conceivable cellular or molecular targets. In-vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax.

The term “affinity” as used herein, refers to the binding strength a binder has to its target. Usually, such affinity is expressed by means of the dissociation constant K_{D}[M], which is an equilibrium constant for the dissociation of an antibody-target complex into its components. It is calculated as the ratio k_{off}/kₒₙ. K_{D} and affinity are inversely related, meaning that a low K_{D} indicates a high affinity, while a high K_{D} indicates a low affinity.

According to other embodiments of the invention, the antibody is a format selected from the group consisting of

IgG

diabody

The IgG format is particularly suited in the context of the preferred way of administration of the fusion protein according to the invention, which is intra-articular. In this preferred way of administration, the fusion protein is not administered systematically, but locally. Hence, possible disadvantages of the IgG format, such as uptake by the liver, which play a role in systemic administration do not count in intra-articular administration.

According to other embodiments of the invention, the antibody, or fragment or derivative thereof retaining target binding properties, and the growth factor, or a fragment or a subunit thereof retaining growth factor activity, are covalently linked by a peptide linker comprising an amino acid sequence selected from any of the group consisting of

a)
(SEQ ID NO: 10)
GGGAKGGGGKAGGGS, called also ″AKKAS″ herein
b)
(SEQ ID NO: 11)
GGGGDGGGGDGGGGS, called also ″DDS″ herein
c)
(SEQ ID NO: 12)
GSADGGSSAGGSDAG, called also ″SAD″ herein,
and
d)
(SEQ ID NO: 13)
GGGGSGGGGEGGGGS, called also ″SES″ herein
e)
(SEQ ID NO: 14)
GGGGSGGGGSGGGGS, called also ″(G4S)3″ herein

The inventors have for the first time used these linkers in fusion peptides of the above kind.

The “AKKAS” linker is positively charged. The “DDS” linker is negatively charged. The “SAD” linker contains both positive and negative charges, the “SES” linker is partially negatively charged, the “(G4S)3” has a neutral charge.

As shown in detail in Example 1 and Table 4, the inventors found that said five linker peptides a) to e) allow the production of the fusion protein when used to genetically conjugate a growth factor to the N-terminus of an antibody.

When said peptide linkers having amino acid sequences selected from any of the group consisting of linkers a), b), c), d) or e), were placed at the N-terminus of the antibody the resulting fusion proteins were also found not to be compromised in terms of activity, efficacy or stability.

In one embodiment, the antibody has the following CDRs:

| | |
|---|---|
| VL-CDR1 | SEQ ID NO: 1 |
| VL-CDR2 | SEQ ID NO: 2 |
| VL-CDR3 | SEQ ID NO: 3 |
| VH-CDR1 | SEQ ID NO: 4 |
| VH-CDR2 | SEQ ID NO: 5 |
| VH-CDR3 | SEQ ID NO: 6 |

In a preferred embodiment thereof, the antibody has a light chain variable domain (VL) according to SEQ ID NO: 7 and a heavy chain variable domain (VH) according to SEQ ID NO: 8.

It is noted that the CDR and VH/VL sequences can be subject of slight variances, encompassing three or less amino acid substitutions, deletions, or insertions, while still maintaining target-binding capacities. Hence, it is further provided that sequences which are 90% identical, preferably 93, 95, 98 or 99% identical, are also encompassed by the scope of the invention.

The slight variances may be made in one or more framework regions and/or one or more CDRs. Three, two or one amino acid substitutions may be made within the framework region of the VH and/or VL domain. For example, one amino acid substitution may be made within the framework region of the VH at position 47 of SEQ ID NO: 8. In some embodiments, the glutamine (Gln or Q) at said position may be substituted with a different amino acid, preferably tryptophan (Trp or W).

The CDRs and VL/VH domains set forth above stem from the monoclonal antibody C11, which is disclosed in WO2016/016269. C11 is defined, in its broadest fashion, by its CDRs. Individual CDRs can be defined according to Kabat [Kabat et al (1991)] or according to Chothia [Chothia and Lesk (1987)] numbering system or both. The definition of VH-CDR1 in WO2016/016269 is according to Chothia. One preferred embodiment of C11 is defined by its VL/VH sequences comprising the said CDRs. In one particularly preferred embodiment, C11 is an IgG comprising said VL/VH sequences. The content of WO2016/016269 is incorporated by reference herein, in particular with respect to C11, and alternatives to C11 that still bind collagen.

Hence, in one preferred embodiment of the present invention the antibody is C11.

In still another aspect of the present invention, the recombinant fusion protein comprises a growth factor, or fragment or subunit thereof, which is insulin-like growth factor (IGF). Preferably, this insulin-like growth factor is human IGF. Likewise preferably, this insulin-like growth factor is IGF-1.

According to yet another aspect, the invention, provides an antibody, or a fragment or derivative thereof retaining target binding properties, and a growth factor, or a fragment or a subunit thereof retaining growth factor activity, wherein said antibody, or fragment or derivative thereof, and said growth factor, or fragment or subunit thereof, are linked by a peptide linker, and wherein the linker comprises an amino acid sequence selected from any of the group consisting of

a)
(SEQ ID NO: 10)
GGGAKGGGGKAGGGS
b)
(SEQ ID NO: 11)
GGGGDGGGGDGGGGS
c)
(SEQ ID NO: 12)
GSADGGSSAGGSDAG
d)
(SEQ ID NO: 13)
GGGGSGGGGEGGGGS,
and
e)
(SEQ ID NO: 14)
GGGGSGGGGSGGGGS.

In this embodiment, the antibody can be fused, via the linker, to the C-terminus of the growth factor, or vice versa. Likewise, the scope of this invention is not limited to embodiments where the antibody binds collagen, nor where the growth factor is insulin-like growth factor. The arguments and preferred embodiments set forth above apply here as well. This applies, inter alfa, to advantages in production yield.

According to yet another aspect, the invention, provides an antibody, or a fragment or derivative thereof retaining target binding properties, and a growth factor, or a fragment or a subunit thereof retaining growth factor activity, wherein said antibody, or fragment or derivative thereof, and said growth factor, or fragment or subunit thereof, are linked by a peptide linker, wherein said antibody has the following CDRs

| | |
|---|---|
| VL-CDR1 | SEQ ID NO: 1 |
| VL-CDR2 | SEQ ID NO: 2 |
| VL-CDR3 | SEQ ID NO: 3 |
| VH-CDR1 | SEQ ID NO: 4 |
| VH-CDR2 | SEQ ID NO: 5 |
| VH-CDR3 | SEQ ID NO: 6, |

and wherein said growth factor, or fragment or subunit thereof, is insulin-like growth factor (IGF).

In this embodiment, the antibody can be fused, via the linker, to the C-terminus of the growth factor, or vice versa. Likewise, the scope of this invention is not limited to embodiments where the linker is any of SEQ ID NO: 10-14. The arguments and preferred embodiments set forth above apply here as well. This applies, inter alfa, to the efficacy of the antibody, and the synergistic interplay between the antibody and the growth factor.

The arguments and preferred embodiments set forth above apply here as well.

The antibody/growth factor ratio can preferably be as shown in Table 1:

TABLE 1
preferred antibody/growth factor ratios

| | | | antibody/ |
|---|---|---|---|
| | | | growth |
| Type | example | conformation | factor ratio |
| Double chain | IgG, F(ab)2 | 1 growth factor/per HC or LC | 1:2 |
| | | 1 growth factor per HC and LC | 1:4 |
| | | Only one HC has a growth factor | 1:1 |
| Single chain | ScFv, Fab | 1 growth factor per HC and LC | 1:2 |
| | | 1 growth factor per HC or LC | 1:1 |

In some embodiments of the invention, the fusion peptide has the following elements (Table 2):

TABLE 2
embodiments of the invention

| Antibody | Peptide linker | Payload |
|---|---|---|
| IgG, scFv or other | SEQ ID NO: 10-14 | growth factor |
| format | | |
| Anti-collagen antibody | SEQ ID NO: 10-14 | IGF |
| Anti-collagen I and | SEQ ID NO: 10-14 | IGF-1 |
| II antibody | | |
| Anti-human-collagen | SEQ ID NO: 10-14 | human IGF-1 (e.g., |
| I and II antibody | | SEQ ID NO: 9, or |
| having CDRs SEQ | | sequences having > |
| ID NO: 1-6 (or | | 90% identify therewith) |
| sequences having > | | |
| 90% identify therewith) | | |
| Anti-human-collagen | SEQ ID NO: 10-14 | human IGF-1 (e.g., |
| I and II antibody | | SEQ ID NO: 9, or |
| having VL/VH SEQ | | sequences having > |
| ID NO: 7-8 (or | | 90% identify therewith) |
| sequences having > | | |
| 90% identify therewith) | | |
| Anti-human-collagen | SEQ ID NO: 10-14 | human IGF-1 (e.g., |
| I and II antibody C11 | | SEQ ID NO: 9, or |
| | | sequences having > |
| | | 90% identify therewith) |

According to an embodiment of the invention, the recombinant fusion protein comprises IGF-1, a peptide linker having any of the sequences a), b), c), d) or e) that is placed between the C terminus of the IGF-1 molecule and the N terminus of the C11 antibody, and the C11 antibody.

According to another embodiment of the invention, the recombinant fusion protein comprises IGF-1, a peptide linker having any of the sequences a), b), c), d) or e) (SEQ ID NOs 10-14) that is placed between the C terminus of the IGF-1 molecule and the N terminus of the C11 antibody, and the C11 antibody, wherein the C11 antibody has an IgG format or a diabody format.

According to another embodiment of the invention, the recombinant fusion protein comprises IGF-1, the peptide linker is (G45)3 or AKKAS and is placed between the C terminus of the IGF-1 molecule and the N terminus of the C11 antibody, and the C11 antibody, wherein the C11 antibody has an IgG format or a diabody format.

Preferably, the fusion protein according to the invention is a recombinant fusion protein.

In one embodiment, the fusion protein comprises at least one chain comprising the amino acid sequence of any of SEQ ID NO: 15, 16, 17, 18, 19, 20.

Each of these sequences comprises the amino acid sequences, in N->C direction, of IGF-1 (SEQ ID NO: 9), the linker according to the above description (SEQ ID NO: 10, 11, 12, 13 or 14), and the variable heavy chain of C11 (SEQ ID NO: 8).

Note that, like the CDR and VL/VH sequences, the sequence of IGF-1 given in SEQ ID NO: 9 can vary, because there exist different isotypes and mutants of IGF-1 which all maintain their physiological activity.

Hence, it is further provided that functional IGF-1 variants having sequences which are 90% identical, preferably 93, 95, 98 or 99% identical to SEQ ID NO: 9, are also encompassed by the scope of the invention.

In one other embodiment, the fusion protein comprises two of the said chains, plus two antibody light chains. See FIG. 9 for an illustration of this embodiment.

According to one further aspect of the present invention, said fusion proteins can be generated by a method of production of a fusion protein comprising the following steps:
a) cloning of genomic, synthetic or complementary (c)DNA encompassing nucleic acid sequences encoding (i) said antibody, or fragment or derivative thereof, (ii) said growth factor, or fragment or subunit thereof, and (iii) said peptide linker, into at least one expression vector suitable for expression of a respective fusion protein,
b) expression of said fusion protein in a suitable expression system, and
c) purification of said fusion protein.

In one embodiment of the present invention, said expression system comprises a mammalian cell line. Said cell line is preferably a Chinese Hamster Ovary (CHO) cell line.

In one embodiment of the present invention, said method of production yields 10 mg fusion protein per liter cell culture or more.

According to one further aspect of the present invention, the use of a fusion protein according to the above description for medical treatment is provided.

According to still one further aspect of the present invention, a fusion protein according to the above description for use as a medicament is provided.

According to one further aspect of the present invention, a method of treating a human subject or animal subject, preferably a horse or a dog, which is suffering from, at risk of developing, or diagnosed for (a) arthritis, preferentially osteoarthritis, or (b) ligament or tendon injury, is provided, which method comprises administering to said subject a fusion protein according to the above description.

According to one further aspect of the present invention, a fusion protein according to the above description is provided for use in the treatment of a human or animal subject which is suffering from, at risk of developing, or diagnosed for, (a) arthritis, preferentially osteoarthritis, or (b) ligament or tendon injury

In one particularly preferred embodiment, said animal is a mammal, preferably a horse or a dog. Both species do regularly suffer from such diseases. The sequence of most growth factors is similar in most mammals, including human, dog and horse. Specifically, IGF1 (SEQ ID NO: 9) is identical in human, dog and horse.

As discussed above, that in such case the antibody can be mammalized, e.g., caninized or equinized, as discussed above.

In one embodiment, the fusion protein is administered by an intra-articular injection. For this way of administration, the fusion protein has to be administered in high concentrations, demanding a suitable formulation. See example 5-8, which show that the fusion proteins according to the invention can be stably formulated in high concentrations.

According to another aspect of the invention, an ex vivo method of pre-treatment of ligaments or tendons used for reparative surgery is provided, which method comprises incubating a ligament or tendon with a fusion protein according to the above description.

Preferably, the ligaments are cruciate ligaments, while the tendons are patellar tendons. Reference is made to example 13 in this context.

The term “fusion protein” as used according to the present invention relates to chimeric proteins created through the joining of two or more nucleic acid sequences which are derived from different genes that originally coded for separate proteins, or different parts of a gene that originally coded for different regions or domains of a protein.

The term “expression vector” according to the present invention refers to a genetic vector comprising at least an expression cassette.

The term “expression cassette” relates to a nucleic acid molecule and a region of a nucleic acid molecule, respectively, containing a regulatory element or promoter being positioned in front of the coding region, a coding region and an open reading frame, respectively, as well as a transcriptional termination element lying behind the coding region. The regulatory element and the promoter, respectively, residing in front of the coding region, can be a constitutive, i.e., a promoter permanently activating the transcription (e.g., CMV promoter), or a regulatable promoter, i.e., a promoter which can be switched on and/or off (e.g., a tetracycline regulatable promoter). The coding region of the expression cassette can be a continuous open reading frame as in the case of a cDNA having a start codon at the 5′ end and a stop codon at the 3′ end. The coding region can be comprised of a genomic or a newly combined arrangement of coding exons and interspersed non-coding introns. However, the coding region of the expression cassette can be comprised of several open reading frames, separated by so-called IREs (Internal Ribosome Entry Sites).

As used herein, the term transfection means the introduction of foreign DNA into the nucleus of eukaryotic cells, or of RNA into eukaryotic cells. Transfection can be mediated by various methods including, but not limited to, calcium phosphate precipitation, DEAE-dextran method, the use of lipids, liposomes, cationic polymers, activated dendrimers, or magnetic beads, Nucleofector™ technology, electroporation, microinjection, “gene gun” technologies or viral vector-based transfer.

In stable transfection, foreign DNA is delivered to the nucleus by passage through the cell and nuclear membranes, is integrated into the host genome, and is sustainably expressed.

In transient transfection, foreign DNA is delivered into the nucleus of eukaryotic cells but is not integrated into the genome, or foreign RNA is delivered into the cytosol where it is translated. Gene expression is usually limited to a certain period of time in transient transfection; in proliferating cells, the transfected nucleic acid is getting diluted out over time.

As used herein, the term “cell line” refers to cells which are genetically modified in such a way that they may continue to grow permanently in cell culture under suitable culture conditions. Such cells can be immortalized cells or transformed cells.

### EXAMPLES

Additional details, features, characteristics and advantages of the object of the invention are disclosed in the dependent claims, and the following description of the respective examples and figures, which, in an exemplary fashion, show preferred embodiments of the present invention. However, these drawings should by no means be understood as to limit the scope of the invention.

### Example 1: Production of IGF1-C11 Fusion Proteins by Transient Gene Expression in CHO-S Cells

### (A) Cloning of Fusion Proteins Comprising IGF1 and Anti-Collagen-II Antibody C11

The genes encoding the antibody fusion proteins comprising IGF-1 (from Homo sapiens) and anti-collagen-II antibody C11 were generated using PCR assembly. The sequence encoding IGF-1 (lacking the signal peptide sequence) was linked via 5 different sequences encoding 15 amino acids (GGGAK-GGGGK-AGGGS, SEQ ID NO: 10); (GSADG-GSSAG-GSDAG, SEQ ID NO: 12); (GGGGS-GGGGE-GGGGS, SEQ ID NO: 13); (GGGGD-GGGGD-GGGGS, SEQ ID NO: 11); GGGGS-GGGGS-GGGGS (SEQ ID NO: 14) (Table 3) to the N-terminus of the gene fragment encoding the variable region of the heavy chain of the C11 antibody (C11(VH)). A sequence encoding an IgG-derived signal peptide was added at the N-terminus to enable high yield production of the encoded fusion proteins. Using engineered HindIII and XhoI restriction sites, the different PCR assembled fragments comprising the “signal peptide-IGF-1-15 amino acids linker-C 11(VH)” were cloned into the pMM137-C11(IgG) vector carrying the full- length heavy and light chain genes of the C11 antibody. A schematic illustration of the assembled genes is shown in FIG. 1.

The amino acid sequences of the mature C11-variable light chain and IGF-1-C11 variable heavy chain fusion proteins employed in the experiments reported below are shown in SEQ ID NOs: 7 and 8 respectively. The signal peptides are cleaved after expression of the fusion proteins and thus are not part of the mature fusion proteins.

TABLE 3
Peptidic linkers used to fuse the human IGF1
sequence to the N-terminus of the C11 heavy
chain

| Peptidic linker | ″short name″ |
|---|---|
| GGGAK-GGGGK-AGGGS (SEQ ID NO: 10) | AKKAS |
| GSADG-GSSAG-GSDAG (SEQ ID NO: 12) | SAD |
| GGGGS-GGGGE-GGGGS (SEQ ID NO: 13) | SES |
| GGGGD-GGGGD-GGGGS (SEQ ID NO: 11) | DDS |
| GGGGSGGGGSGGGGS (SEQ ID NO: 14) | (G4S)3 |

### (B) Expression of Fusion Proteins

Fusion proteins comprising IGF-1 fused to the C11 IgG by means of different linkers were produced by transient gene expression in suspension-adapted CHO-S cell cultures. Cells were expressed transiently at 0.5 L scale via PEI-mediated transfection. Following transfection cells were maintained in PowerCHO-2 medium (supplemented with 4 mM Ultraglutamine) for 6 days at 31° C. under shaking conditions, after which the culture supernatant was harvested by centrifugation and further processed to purify the fusion protein.

### (C) Purification of Fusion Proteins Using Protein A Resin

Transfected CHO-S cell suspension cultures were centrifuged for 30 minutes at 5000 rpm at 4° C. The supernatant was further clarified by filtration using 0.45 μm filters (rapid Flow Bottle Top filters, Nalgene). Protein A resin (Ultra linked Protein A resin, Sino Biological Inc.) was added to the filtered supernatant and the mixture incubated under shaking conditions for ca. 1 h. The resin was then collected into a liquid chromatography column (SIGMA), and washed with “buffer A” (100 mM NaCl, 0.5 mM EDTA, 0.1% Tween 20 in PBS pH 7.4) followed by a second wash with “buffer B” (500 mM NaCl, 0.5 mM EDTA in PBS pH 7.4). The fusion proteins were eluted by gravity flow using 0.1 M glycine, pH3. Aliquots were collected and fractions containing the fusion protein, as confirmed by UV spectrometry, were pooled and dialyzed overnight against PBS.

### (D) Results

The five different variants of the IGF-1-C11 fusion proteins were expressed in CHO-S cells at 500 mL scale by transient gene expression. An expression experiment was performed leading to the purification of a protein batch. Following transfection with the corresponding mammalian expression vectors, cells were maintained for 6 days at 31° C. under shaking conditions. The supernatant was harvested by centrifugation and 0.4 μm filtration and the fusion proteins were purified by Protein-A affinity chromatography. The variants showed improved volumetric yields of expression in a transient gene expression experiment (Table 4).

TABLE 4
Results of the expression test performed in two batches
(“A” and “B”); volumetric yields of the five IGF-1-C11
variants upon transient gene expression

| | | Yields | | Yields |
|---|---|---|---|---|
| | | after | | after |
| | | elution | | elution |
| Protein | Batch | (mg/L) | Batch | (mg/L) |
| AKKAS | A | 24.8 | B | 28.2 |
| SAD | A | 10 | B | 17.6 |
| SES | A | 17 | B | 23.2 |
| DDS | A | 10.4 | B | 19.2 |
| (G4S)3 | A | 10.2 | B | 18.6 |

### Example 2: Characterization of the IGF-1-C11 Fusion Proteins by SDS-PAGE and Western Blot Analysis

### (A) SDS-PAGE and Western Blot Analysis

5-μg-aliquots of the different purified fusion proteins were analyzed under reducing and non-reducing conditions using SDS-PAGE followed by Coomassie Blue staining. For Western blot analysis, samples were run in 4-12% SDS-PAGE, transferred to Immobilon-P membranes and detected using rabbit anti-human IGF1 (1:500) followed by an anti-rabbit-IgG-HRP (1:2′000). Band signals were visualized on X-ray film using chemiluminescence ECL detection reagents.

### (B) Results

Integrity of the fusion proteins was analysed by SDS-PAGE followed by Coomassie Blue staining (FIG. 2). All the variants showed bands at the expected molecular weight under reducing or non-reducing conditions, suggesting that all the linker variants are suitable for the expression of the IGF-1-C11 fusion protein.

Identity and integrity of the fusion proteins were analysed by Western blot using rabbit anti-human IGF-1 antibody (FIG. 3). Under reducing conditions only a single band corresponding to IGF-1 fused to the heavy chain of the C11 antibody (IGF-1-HC(C11)) could be detected. Under non-reducing conditions beside the main band of ca. 160 KDa corresponding to the fully assembled IGF-1-C11 fusion protein, two smaller bands probably corresponding to IGF-1-C11 missing one copy of the light chain (expected size 137.4 KDa), and IGF-1-C11 missing both copies of the light chain (expected size 114 KDa), could be detected. Under these experimental conditions no cleavage of the IGF-1 molecule from the C11-heavy chain, or other degradation products could be detected in any of the IGF-1-C11 variants.

### Example 3: Characterization of the IGF-1-C11 Fusion Proteins by Size Exclusion Chromatography (SEC)

### (A) Size Exclusion Chromatography of Fusion Proteins

Size exclusion chromatography of fusion proteins was performed using a Superdex 200 increase 10/300 GL column (GE Healthcare) with PBS as running buffer on an ÄKTA-FPLC system (GE healthcare). 100 μl protein solutions at a concentration of 0.25 mg/mL were injected into a loop and automatically injected onto the column. UV absorbance at 280 nm was assessed over time.

### (B) Results

Homogeneity and aggregate state of the conjugate preparations were analysed by size exclusion chromatography using a Superdex S200 Increase 10/300 GL column (FIG. 4). All the IGF-1-C11 variants showed a main peak at a retention volume of about 11.4 mL, which is in line with the expected molecular weight of the protein, and additional minor peaks representing protein aggregates eluting at earlier retention volumes.

### Example 4: Characterization of the IGF1-C11 Fusion Proteins by Surface Plasmon Resonance (Biacore)

### (A) Surface Plasmon Resonance (BIAcore) of Fusion Protein

The binding affinity to Collagen-II of fusion proteins comprising IGF1 and the C11 antibody was analyzed using surface plasmon resonance (Biacore X-100 system, GE Healthcare). A microsensor chip (CM5, GE Healthcare) was coated with Collagen-II with ca 2000 resonance units coating density. For analysis on surface plasmon resonance, proteins were filtered with a syringe driven filter unit (Millex®-GV, Low protein binding durapore membrane, 0.22 μm) and their concentration determined with a spectrophotometer. The different fusion proteins were injected at concentrations of 800, 400, 200, and 100 nM.

### (B) Results

After fusion with IGF1, using different peptidic linkers, the binding capacity of the C11 moiety to Collagen-II was maintained, as confirmed using surface plasmon resonance (BIAcore). As shown in FIG. 5 the different fusion proteins retained the ability to bind to Collagen-II with comparable binding kinetic.

### Example 5: Concentration of the IGF1-C11 Fusion Proteins

### (A) Concentration of the IGF1-C11 Fusion Proteins

Fusion proteins comprising IGF1 fused to the C11 antibody by meaning of different peptidic linkers, were produced by transient gene expression in suspension adapted CHO-S cell cultures. Following purification by Protein-A and dialysis against PBS the different samples were concentrated to 10 mg/mL in PBS using Vivaspin® Turbo 15 ultrafiltration spin columns (Sartorius Stedim, MWCO 10 KDa). The optical density of the samples was than determined using a spectrophotometer (OD280 nm) and used to assess the final concentration of the samples.

For SDS-PAGE analysis ca 5 ug aliquots of the different fusion proteins, before and after ultrafiltration, were analyzed under reducing conditions using 4-12% SDS-PAGE gels followed by Coomassie Blue staining.

Size exclusion chromatography of fusion proteins was performed using a Superdex 200 increase 10/300 GL column (GE Healthcare) with PBS as running buffer on a AKTA-FPLC system (GE healthcare). For the analysis samples were diluted to 0.25 mg/mL (samples before ultrafiltration) or 0.4 mg/mL (samples after ultrafiltration) and 100 μl protein solutions were injected into a loop and automatically injected onto the column. UV absorbance at 280 nm was assessed over time.

### (B) Results

The different fusion variants could be formulated at ca 10 mg/mL in PBS as confirmed by SEC and SDS-PAGE analysis (Table 5, FIG. 6), this demonstrate a good solubility of the different IGF1 based fusion proteins.

TABLE 5
Formulation of the different IGF1-C11 variants at ca 10 mg/mL in
PBS. Following ProteinA purification, protein samples produced by TGE
were concentrated using Vivaspin ® Turbo 15 ultrafiltration spin
columns (Sartorius Stedim, MWCO 10 KDa).
Formulation studies

| | | Concentration | | |
|---|---|---|---|---|
| | OD280 | before | | Concentration |
| IGF1-C11 | before | ultrafiltration | OD280 after | after |
| variants | ultrafiltration | (mg/mL) | ultrafiltration | ultrafiltration |
| (G4S)3 | 0.431 | 0.36 | 12.590 | 10.49 |
| AKKAS | 0.584 | 0.49 | 12.509 | 10.42 |
| SAD | 0.505 | 0.42 | 12.071 | 10.06 |
| SES | 0.548 | 0.46 | 12.035 | 10.03 |
| DDS | 0.461 | 0.38 | 12.023 | 10.02 |

### Example 6: Stability Studies of the IGF1-C11 Fusion Proteins uring the Freezing Storage

### (A) Freeze-Thaw Stability

The 5 fusion proteins comprising IGF1 fused to the C11 antibody by meaning of different peptidic linkers were concentrated to 10 mg/mL and subjected to 4 cycles of freeze and thaw in order to determine protein stability upon freezing storage. Protein samples were snap frozen by plunging the vials into liquid Nitrogen for about 2 minutes, frozen samples were than incubated for about 5 minutes at room temperature till samples were completely thawed. The freeze and thaw procedure was repeated for a total of 4 times after which the samples were analyzed for the presence of aggregates or degraded fragments by OD measurement at 280 nm, Size exclusion Chromatography, SDS-PAGE and Western Blotting.

The optical density of the samples was determined using a spectrophotometer (OD280 nm). Size exclusion chromatography of fusion proteins was performed using a Superdex 200 increase 5/150 GL column (GE Healthcare) with PBS as running buffer on a AKTA-FPLC system (GE healthcare). 20 μl protein solutions at a concentration of ca 10 mg/mL were injected into a loop and automatically injected onto the column. UV absorbance at 280 nm was assessed over time. SDS-PAGE analysis were performed under reducing conditions using ca 5 ug aliquots of the different fusion proteins that were run an 4-12% SDS-PAGE followed by Coomassie Blue staining. For Western blot analysis, samples were run in 4-12% SDS-PAGE, transferred to Immobilon-P membranes and detected using rabbit anti-human IGF1 (1:500) followed by an anti-rabbit-IgG-HRP (1:2′000). Band signals were visualized on X-ray film using chemiluminescence ECL detection reagents.

### (B) Results

Integrity of the fusion proteins after 4 rounds of freeze/and thawing was analyzed by SDS-PAGE followed by Coomassie blue staining (FIG. 7A), Western Blotting (FIG. 7B) and size exclusion chromatography (FIG. 7C). The band profile of the different IGF1-C11 variants in SDS-PAGE and Western Blotting, was comparable at TO and after 4 cycles of freeze and thawing. Whereas the bands corresponding to the IGF1-HC and C11-LC fragments (SDS-PAGE) or the IGF1-HC fragment alone (western Blotting) could be detected by SDS-PAGE and Western Blotting respectively, under these conditions no cleavage of the IGF1 molecule from the C11-heavy chain, or other degradation products could be observed. Similarly, the Size Exclusion profile of the different IGF1-C11 variants was preserved after repeated freeze and thaw cycles with a main peak at a retention volume of about 1.7 mL, which is in line with the expected molecular weight of the protein, and additional minor peaks representing protein aggregates eluting at earlier retention volumes.

### Example 7: Stability Studies of the IGF1-C11 Fusion Proteins DuringStorage

### (A) Stability Studies During Storage

Following concentration to 10 mg/mL, the 5 fusion proteins comprising IGF1 fused to the C11 antibody by meaning of different peptidic linkers, were aliquoted (50 uL aliquots) into 0.5 mL tubes and incubated either at 4° C., 25 ° C. or 37° C. Single aliquots from the 5 different fusion proteins, were analyzed by OD280 measurement, SEC, SDS-PAGE and Western Blotting after 1 days, 1 week and 1 month incubation at the defined temperatures.

The optical density of the samples was determined using a spectrophotometer (OD280nm). Size exclusion chromatography of fusion proteins was performed using a Superdex 200 increase 5/150 GL column (GE Healthcare) with PBS as running buffer on a AKTA-FPLC system (GE healthcare). 20 μl protein solutions at a concentration of ca 10 mg/mL were injected into a loop and automatically injected onto the column. UV absorbance at 280 nm was assessed over time. SDS-PAGE analysis were performed under reducing conditions using ca 5 ug aliquots of the different fusion proteins that were run an 4-12% SDS-PAGE followed by Coomassie Blue staining. For Western blot analysis, samples were run in 4-12% SDS-PAGE, transferred to Immobilon-P membranes and detected using rabbit anti-human IGF1 (1:500) followed by an anti-rabbit-IgG-HRP (1:2′000). Band signals were visualized on X-ray film using chemiluminescence ECL detection reagents.

### (B) Results

Table 6 summarizes the results of the stability study at different temperatures and different time-points of the 5 fusion proteins. OD measurement at 280 nm did not reveal major differences between the different protein variants and incubation conditions. All protein variants showed good stability up to 1 month incubation at 4° C. with neither apparent changes in protein quality nor major signs of degradation as shown by SEC analysis and SDS-PAGE or Western Blotting. Incubation at 25° or 37° C. for at least 1 week resulted in the appearance in the SEC profile of all the samples of minor degradation products (representing ca 0.5-8% of the total peak area) with a retention volume greater than 2.5 mL. This correlated with the appearance, in both SDS-PAGE and Western Blotting, of a new band for the IGF1-HC fragment with an apparent smaller molecular weight than the theoretical one.

TABLE 6
Analysis of the stability of the different IGF1-C11 protein fusions under different
incubation conditions. (°) Variation in OD280 measurement relative to the measurement
at T0. (*) SDS-PAGE analysis under reducing conditions, H = IGF1-HC high molecular
weight band L = IGF1-HC low molecular weight band. (⁺) Western Blot analysis under
reducing conditions using an anti-IGF1 antibody, H = IGF1-HC high molecular weight
band L = IGF1-HC low molecular weight band. (§) The relative amount of the aggregates,
main peak, and degradation fragments has been reported as percentage of the total area
of the peaks.
Freeze-Thaw and Storage Stability Studies

| | | | Methods | | | | | |
|---|---|---|---|---|---|---|---|---|
| | | | | | | SEC (§) | | |
| IGF1- | | | Δ OD | | | | Main | Degradation |
| C11 | | Time | 280 | SDS- | Western | Aggregates | peak | fragments |
| variants | Temperature | point | (%) (°) | PAGE (*) | Blot (⁺) | (%) | (%) | (%) |
| (G4S)3 | | T0 | 0 | H | H | 13.18 | 86.82 | — |
| (G4S)3 | | F/T | −1.4 | H | H | 20.44 | 79.56 | — |
| (G4S)3 | +4° C. | 1 day | 0.1 | H | H | 19.71 | 80.29 | — |
| (G4S)3 | +4° C. | 1 week | 1.3 | H | H | 19.93 | 80.07 | — |
| (G4S)3 | +4° C. | 1 month | 1.3 | H | H | 19.63 | 79.6 | 0.78 |
| (G4S)3 | +25° C. | 1 day | 3.2 | H | H | 20.17 | 79.83 | — |
| (G4S)3 | +25° C. | 1 week | 3.6 | L | H/L | 13.92 | 86.07 | — |
| (G4S)3 | +25° C. | 1 month | 0.1 | L | L | 6.34 | 88.26 | 5.4 |
| (G4S)3 | +37° C. | 1 day | 3.5 | H | H | 22.63 | 77.37 | — |
| (G4S)3 | +37° C. | 1 week | 15.2 | L | H/L | 18.33 | 79.67 | 2 |
| (G4S)3 | +37° C. | 1 month | 0.8 | L | L | 10.18 | 84.05 | 5.77 |
| AKKAS | | T0 | 0 | H | H | 20.18 | 79.81 | — |
| AKKAS | | F/T | −1.4 | H | H | 21.78 | 78.23 | — |
| AKKAS | +4° C. | 1 day | −0.1 | H | H | 21.54 | 78.46 | — |
| AKKAS | +4° C. | 1 week | −0.3 | H | H | 21.15 | 78.84 | — |
| AKKAS | +4° C. | 1 month | 2.3 | H | H | 19.78 | 79.77 | 0.45 |
| AKKAS | +25° C. | 1 day | 4.5 | H | H | 21.97 | 78.03 | — |
| AKKAS | +25° C. | 1 week | 6.6 | H | H | 21.41 | 78.59 | — |
| AKKAS | +25° C. | 1 month | 3.5 | H/L | H/L | 16.24 | 81.92 | 1.84 |
| AKKAS | +37° C. | 1 day | 6.1 | H | H | 23.75 | 76.25 | — |
| AKKAS | +37° C. | 1 week | 26.6 | H/L | H/L | 22.68 | 75.2 | 2.12 |
| AKKAS | +37° C. | 1 month | 7.4 | L | L | 9.39 | 84.83 | 5.78 |
| SAD | | T0 | 0 | H | H | 14.72 | 85.28 | — |
| SAD | | F/T | 0.1 | H | H | 15.49 | 84.51 | — |
| SAD | +4° C. | 1 day | 3.7 | H | H | 15.78 | 84.22 | — |
| SAD | +4° C. | 1 week | 4.4 | H | H | 16.51 | 83.49 | — |
| SAD | +4° C. | 1 month | 2.0 | H | H | 13.28 | 85.94 | 0.77 |
| SAD | +25° C. | 1 day | 4.8 | H | H | 15.8 | 84.2 | — |
| SAD | +25° C. | 1 week | 5.1 | L | H/L | 9.09 | 90.91 | — |
| SAD | +25° C. | 1 month | 2.0 | L | L | 3.57 | 91.52 | 4.91 |
| SAD | +37° C. | 1 day | 4.7 | H | H | 17.07 | 82.93 | — |
| SAD | +37° C. | 1 week | 8.4 | L | H/L | 10.26 | 89.04 | 0.69 |
| SAD | +37° C. | 1 month | 3.6 | L | L | 4.28 | 89.93 | 5.78 |
| SES | | T0 | 0 | H | H | 18.91 | 81.09 | — |
| SES | | F/T | −1.0 | H | H | 20.87 | 79.13 | — |
| SES | +4° C. | 1 day | 3.5 | H | H | 20.68 | 79.32 | — |
| SES | +4° C. | 1 week | 3.2 | H | H | 21.36 | 78.64 | — |
| SES | +4° C. | 1 month | 1.9 | H | H | 19.83 | 79.27 | 0.9 |
| SES | +25° C. | 1 day | 4.9 | H | H | 21.49 | 78.51 | — |
| SES | +25° C. | 1 week | 4.8 | H/L | H/L | 21.23 | 78.77 | — |
| SES | +25° C. | 1 month | 2.6 | H/L | H/L | 20.17 | 77.57 | 2.26 |
| SES | +37° C. | 1 day | 5.1 | H | H | 21.42 | 78.58 | — |
| SES | +37° C. | 1 week | 8.9 | H/L | H/L | 25.08 | 74.42 | 0.49 |
| SES | +37° C. | 1 month | 8.6 | L | L | 6.11 | 85.61 | 8.28 |
| DDS | | T0 | 0 | H | H | 13.56 | 86.44 | — |
| DDS | | F/T | −1.8 | H | H | 15.97 | 84.03 | — |
| DDS | +4° C. | 1 day | 0.3 | H | H | 15.99 | 84.01 | — |
| DDS | +4° C. | 1 week | 1.2 | H | H | 16.05 | 83.95 | — |
| DDS | +4° C. | 1 month | 1.3 | H | H | 15.12 | 83.93 | 0.95 |
| DDS | +25° C. | 1 day | 2.3 | H | H | 17.03 | 82.97 | — |
| DDS | +25° C. | 1 week | 2.3 | H/L | H/L | 13.67 | 86.33 | — |
| DDS | +25° C. | 1 month | 1.3 | H/L | H/L | 13.92 | 84.52 | 1.56 |
| DDS | +37° C. | 1 day | 2.2 | H | H | 17.29 | 82.71 | — |
| DDS | +37° C. | 1 week | 5.1 | H/L | H/L | 18.91 | 80.41 | 0.68 |
| DDS | +37° C. | 1 month | 1.2 | L | L | 7.11 | 88.61 | 4.29 |

### Example 8: Stability Studies of the IGF1-C11 Fusion Proteins in Human Serum

### (A) Serum tability

Aliquots of the different IGF1-C11 fusion proteins were incubated at 37° C. with human serum at a 1:10 ratio for 24 or 120 h. Samples were than run in 4-12% SDS-PAGE, transferred to Immobilon-P membranes and detected using rabbit anti-human IGF1 (1:500) followed by an anti-rabbit-IgG-HRP (1:2′000). Band signals were visualized on X-ray film using chemiluminescence ECL detection reagents.

### (B) Results

Integrity of the different fusion proteins upon incubation with human serum was analysed by Western blot using rabbit anti-human IGF1 antibody (FIG. 8). Under reducing conditions only a single band of ca 57 Kda corresponding to IGF1 fused to the heavy chain of the C11 antibody (IGF1-HC(C11)) could be detected. Under these experimental conditions no cleavage of the IGF1 molecule from the C11-heavy chain, or other degradation products could be detected in none of the IGF -C11 variants.

### Example 9: Preparation and Characterization of IGF1 Conjugated to the Anti-Collagen Antibody C11 in scFv-Fc Format and SIP Format

### (A) Cloning of Fusion Proteins Comprising IGF1 and Anti-Collagen Antibody C11 in Different Antibody Formats

The genes encoding the antibody fusion proteins comprising IGF1 (from Homo sapiens) and anti-Collagen-I and II antibody C11 in different antibody formats were generated using PCR assembly. The sequence encoding IGF1 (lacking the signal peptide sequence) was linked via the 15 amino acid glycine-serine-linker (GGGGS-GGGGS-GGGGS), to the N-terminus of the gene fragment encoding the C11 antibody in scFv-Fc or SIP formats. A sequence encoding an IgG-derived signal peptide was added at the N-terminus to enable high yield production of the encoded fusion proteins.

Using engineered HindIII and Notl restriction sites the assembled PCR fragments corresponding to the IGF1-C11(scFv-Fc) and IGF1-C11(SIP) cDNAs were cloned into the mammalian expression vector pcDNA3.1/Neo(+). The signal peptides were cleaved after expression of the fusion proteins and thus are not part of the mature fusion proteins. A schematic illustration of the assembled genes is shown in FIG. 10.

The amino acid sequence of the IGF1-C11(scFv-Fc) and IGF1-C11(SIP) fusion proteins are shown in SEQ ID NOs 20 and 21, respectively.

### (B) Expression of Fusion Proteins

Fusion proteins comprising IGF1 fused to the C11 antibody in scFv-Fc or SIP format by meaning of the (G45)3 linker, were produced by transient gene expression in suspension adapted CHO-S cell cultures. Cells were expressed transiently at 0.7 to 1 L scale via PEI mediated transfection. Following transfection cells were maintained in PowerCHO-2 medium (supplemented with 4 mM Ultraglutamine), for 6 days at 31° C. under shaking conditions, after which the culture supernatant was harvest by centrifugation and further processed to purify the fusion protein.

(C) Purification of Fusion Proteins Using Protein A Resin

Transfected CHO-S cell suspension cultures were centrifuged for 30 minutes at 5000 rpm at 4° C. The supernatant was further clarified by filtration using 0.45 um filters. Protein A resin was added to the filtered supernatant and the mixture incubated under shaking conditions for ca 1 h. The resin was than collected into a liquid chromatography column, and washed with “buffer A” (100 mM NaCl, 0.5 mM EDTA, 0.1% Tween 20 in PBS pH 7.4) followed by a second wash with “buffer B” (500 mM NaC10.5 mM EDTA in PBS pH 7.4). The fusion proteins were eluted by gravity flow using 0.1 M glycine, pH3. Aliquots were collected and fractions containing the fusion protein, as confirmed by UV spectrometry, were pooled and dialysed overnight against PBS.

### (D) SDS-PAGE and Western Blot Analysis

Aliquots of the different purified fusion proteins were analyzed under reducing and non-reducing conditions using SDS-PAGE followed by Coomassie Blue staining.

### (E) Size Exclusion Chromatography of Fusion Proteins

Size exclusion chromatography of fusion proteins was performed using a Superdex 200 increase 10/300 GL column (GE Healthcare) with PBS as running buffer on a ÄKTA-FPLC system (GE healthcare). 100 μl protein solutions were injected into a loop and automatically injected onto the column. UV absorbance at 280 nm was assessed over time.

### (F) Results: Production of the IGF1-C11 Fusion Proteins in Different Antibody Formats by Transient Gene Expression in CHO-S Cells

The 3 variants of the IGF1-C11 fusion proteins corresponding to IgG, scFv-Fc and SIP formats, were expressed in CHO-S cells at 700 mL to 1 L scale by transient gene expression. Following transfection with the corresponding mammalian expression vectors, cells were maintained for 6 days at 31° C. under shaking conditions. The supernatant was harvested by centrifugation and 0.4 um filtration and the fusion proteins were purified by Protein-A affinity chromatography.

In transient gene expression experiments the IGF1-C11 variant in IgG format showed improved volumetric yields of expression over the scFv-Fc and SIP formats, the latter could not be expressed. (Table 7).

TABLE 7
Results of the volumetric yields obtained by transient gene expression for
the IGF1-C11 fusion protein in different antibody formats. No IGF1-C11
in SIP format could be produced and only a minimal amount of scFv-FC
suggesting the non-obviousness to successfully express such a complex
fusion protein.

| | | | | Yield after |
|---|---|---|---|---|
| | | | | ProteinA |
| | | | Transfection | purification and |
| Protein | Format | Linker | volume (mL) | dialysis(mg/L) |
| IGF1-C11 IgG | IgG | (G4S)3 | 700 | 12.06 |
| IGF1-C11 | scFv-Fc | scFv-Fc (G4S)3 | 700 | 3.94 |
| IGF1-C11SIP | SIP | (G45)3 | 700 | 0 |

### Characterization of the IGF1-C11 Fusion Proteins by SDS-PAGE

Integrity of the fusion proteins was analysed by SDS-PAGE followed by Coomassie blue staining (FIG. 11). Both the IgG and scFv-Fc variants showed bands at the expected molecular weight under reducing (R) on non-reducing (NR) conditions, suggesting that these formats are suitable for the expression of the IGF1-C11 fusion protein.

### Characterization of the IGF1-C11 fusion proteins by Size Exclusion Chromatography (SEC)

Homogeneity and aggregate state of the conjugate preparations was analysed by size exclusion chromatography using a Superdex S200 Increase 10/300 GL column (FIG. 11). In IgG format the fusion protein showed a main peak at a retention volume of ca 11.4 mL, which is in line with the expected molecular weight of the protein. The IGF1-C11(scFv-Fc) format showed a main peak at about 12.3 mL consistent with a smaller molecular weight of the intact protein.

Both fusion protein variants showed additional minor peaks representing protein aggregates eluting at earlier retention volumes.

### Example 10: Preparation and Characterization of IGF1 Conjugated to a Diabody Format of C11

### (A) Cloning and Small Scale Production in Diabody Format

Antibody molecules in whole IgG format and in diabody format were used for conjugation with IGF1. In the case of the whole IgG fusions, IGF-1 was conjugated through the peptide linker (G4S)3 either to the N terminus of the VH according to the cloning scheme depicted in FIG. 12A or to the C terminus of the CH3 according to the cloning scheme depicted in FIG. 12B.

In the case of diabody fusions, IGF-1 was conjugated through the peptide linker (G4S)3 either to the N-terminus of the diabody according to the cloning scheme depicted in FIG. 12C, or to the C terminus of the diabody according to the cloning scheme depicted in FIG. 12D.

In brief, their preparation was prepared as follows: transfected CHO-S cell suspension cultures were centrifuged for 30 minutes at 5000 rpm at 4° C. The supernatant was further clarified by filtration using 0.45 μm filters. Protein A resin was added to the filtered supernatant and the mixture incubated under shaking conditions for ca. 1 h. The resin was then collected into a liquid chromatography column, and washed with “buffer A” (100 mM NaCl, 0.5 mM EDTA, 0.1% Tween 20 in PBS pH 7.4) followed by a second wash with “buffer B” (500 mM NaCl, 0.5 mM EDTA in PBS pH 7.4). The fusion proteins were eluted by gravity flow using 0.1 M glycine, pH3. Aliquots were collected and fractions containing the fusion protein, as confirmed by UV spectrometry, were pooled and dialyzed overnight against PBS.

### (B) Results

The SDS-PAGE, Size Exclusion chromatography and Biacore analysis for IGF1 conjugated to the N-terminus of whole IgG format (C11) is shown in FIGS. 2, 4 and 5. The SDS-PAGE and Size Exclusion chromatography for IGF1 conjugated to the N-terminus of a diabody is shown in FIG. 13A. The SDS-PAGE and Size Exclusion chromatography for IGF1 conjugated to the C-terminus of a diabody is shown in FIG. 13B. These experiments demonstrate that the first embodiment, where the growth factor is conjugated to the N-terminus of the antibody, has superior activity.

### Example 11: In Vitro Activity of IGF1 Conjugated in Different Orientations to Whole IgG's

We tested whether IGF-1 retains its ability to stimulate NIH3T3 cells, which stably express human IGFR-1, when fused to the N-terminus or C-terminus of an irrelevant antibody in whole IgG format with the (G4S)3 linkers. The results are shown in Fig.14A for the fusion at the N-terminus and in FIG. 14B for the fusion at the C-terminus.

The experiments were repeated conjugating IGF-1 to the N-Terminus of C11 both as a whole immunoglobulin G or as a diabody. The results are shown in FIG. 14C for the fusion at the N-terminus of the IgG and in FIG. 14D for the fusion at the N-terminus of the diabody.

A fusion protein having the N->C orientation:

N-Growth factor-Linker-Antibody-C

is utterly different from other immunoconjugates, where the payload, be it a toxin, a growth factor or a cytokine is fused or conjugated to the constant domain (often to the C-Terminus thereof), to not interfere with target binding of the antibody, like, e.g., SS1P, which comprises an anti-mesothelin antibody Fv, the CH1 part of which is linked to the PE38 exotoxin.

The inventors have surprisingly shown that despite these considerations, the fusion of the growth factor-linker to the variable domain of the antibody does not interfere with the target binding of the latter.

### Example 12: In-Vivo Activity of IGF1-C11

### (A) Therapy Experiment

IGF1-C11 (fusion with IgG and the (G4S)3 linker) was tested in a rat medial meniscus tear (MMT) model of osteoarthritis (OA) in comparison with untargeted IGF-1, IGF-1 conjugated to the F8 antibody against the anti-EDA domain of fibronectin and FGF-18, a growth factor which is the benchmark for this model.

For the MMT model of OA, weight matched Lewis rats (300-325 g) were subjected to MMT surgery of the knee. The sham surgery was performed by exposing the joint and transecting the medial collateral ligament. In MMT animals the exposed meniscus was then transected at its narrowest point. The joint and skin were then closed with sutures. After OA was induced by surgery (day 0) the rats were divided in seven different groups (9 rats each) and injected intra-articularly once a week for three weeks (week 3, week 4 and week 5) as follows:

| | | |
|---|---|---|
| 1) PBS | | |
| 2) IGF-1-IgG(KSF) | 1X/week | 600 ug (untargeted IGF-1 fusion protein) |
| 3) IGF-1-IgG(F8) | 1X/week | 600 ug (IGF-1 fusion protein with anti- |
| | | EDA antibody) |
| 4) IGF-1-IgG(C11) | 1X/week | 100 ug |
| 5) IGF-1-IgG(C11) | 1X/week | 300 ug |
| 6) IGF-1-IgG(C11) | 1X/week | 600 ug |
| 7) FGF-18 2X/week | 2X/week | 5 ug |

At week 10 animals were sacrificed and knees harvested for histology. The results of the experiments are shown in FIG. 15.

### (B) Immunohistochemistry

For IHC analysis of tissues taken from the therapeutic studies described in Example 12, all slides were treated with streptavidin block for 15 minutes, biotin block for 15 minutes, Dual Endogenous Enzyme block for 10 minutes and protein block for 20 minutes after epitope retrieval. Subsequent to blocking for endogenous enzyme activity and non-specific binding, Rabbit anti-human antibody at 2 ug/mL was incubated on slides for 30 minutes to detect the primary antibody (C11 or F8). Anti-rabbit HRP polymer was used to label the secondary antibody (10 minutes) followed by application of diaminobenzidine for 2 minutes to stain the reaction. Slides were counterstained with hematoxylin. Three wash steps with wash buffer were performed between each step. The results are shown in FIG. 16.

### Example 13: Staining of Sheep Cruciate Ligaments and Patellar Tendons

### (A) Staining of Cruciate Ligaments and Patellar Tendons by C11 Antibody in IgG Format

Samples of the patellar tendon and the anterior cruciate ligament were obtained from 3 juvenile (approximately 200 days) crossbred domestic sheep which were euthanised following unrelated surgical experimental procedures. The samples were collected within 30 minutes from euthanasia, OCT-embedded and frozen, followed by preservation at −80C. Briefly, purified KSF and C11 antibodies in IgG format were added at the final concentration of 0.5 μg/ml to the sections. Detection of the primary antibody was performed with a rabbit anti-human IgG 1:1000 followed by an anti-rabbit-alexa 488 1:500.

### (B) Staining of Sheep Cruciate Ligaments and Patellar Tendons Using C11 or KSF Fluorescently Labeled with IRDye800CW

Samples of the patellar tendon and the anterior cruciate ligament were obtained from juvenile (approximately 200 days) crossbred domestic sheep which were euthanised following unrelated surgical experimental procedures. The samples were collected within 30 minutes from euthanasia and placed in PBS and moved to 4 C. Both segments were submerged in 1mL of a 50 μg/mL solution of either C11 or KSF fluorescently labeled with IRDye800CW. After gentle mixing, the segments were removed from the labeled solution and repeatedly washed with fresh PBS. Images were acquired on an IVIS Spectrum imaging system (exposure 0.2 s, binning factor 4, excitation at 745 nm, emission filter at 800 nm, f number 2, field of view 13.1). Images were acquired either immediately after the first washing steps (t=0) or after 2 hours (t=2 h). Segments were kept in fresh PBS solution between acquisitions.

### (C) Results

The staining performed with the C11 antibody in IgG format showed a significantly brighter signal compared to KSF (FIG. 17A). A specific and long lasting binding to ligament and cruciate could be observed for the C11 antibody fluorescently labeled with IRDye800CW when compared to the irrelevant KSF antibody (FIG. 17B).

These experiments suggest that a fusion protein comprising a growth factor and a suitable antibody can be used for
treating a patient suffering from, at risk of developing, or diagnosed for ligament or tendon injury, and/or
ex vivo pre-treatment of ligaments or tendons used for reparative surgery.

### Example 14: Measurements of Blood Concentration of IGF1-C11 and IGF1-F9

### (A) Measurements of Blood Concentration

IGF1-C11 (fusion with IgG and the (G4S)3 linker) and IGF1-F9 (fusion with IgG and the (G4S)3 linker) were tested in a rat medial meniscus tear (MMT) model of osteoarthritis (OA) in comparison with IGF1-KSF as negative control. Rats were injected intra-articularly once a week for three weeks with various quantities: 100, 300 or 600 ug/dose and took down on day 42. Blood level of IGF1-C11 and IGF1-F9 after the first two injections were compared to the blood level of IGF1-KSF.

### (B) Results

IGF1-C11 had the lowest blood exposure as compared to IGF1-F9 and IGF1-KSF. The significant exposure difference from IGF1-KSF indicated the effective targeting of fusion via C11 at the site of disease and limited leakage in the blood (FIG. 18A).

IGF1-F9 showed comparable blood exposure as compared to IGF1-KSF (FIG. 18B). The results demonstrate a superiority of the C11 antibody over the F9 antibody as confirmed by its reduced serum concentration indicating higher uptake and retention at the lesion site.

### FIGURES

FIG. 1: Schematic representation of the IGF1-C11 mammalian expression vectors used for the production of the different IGF1-C11 variants in CHO-S cells. SP=signal peptide, C11-LC=light chain sequence of the C11 antibody, pA=polyA signal; C11-VH=variable domain of the heavy chain of the C11 antibody, C11-Fc=C11 heavy chain fragment including the CH1 and

Fc portions of the antibody.

FIG. 2: SDS-PAGE analysis of the IGF1-C11 variants produced by transient gene expression.

FIG. 3: Western Blot analysis of IGF1-C11 variants produced by transient gene expression.

FIG. 4: Size Exclusion Chromatography analysis of the IGF1-C11 variants produced by transient gene expression.

FIG. 5: Surface Plasmon Analysis (BlAcore) of the different IGF1-C11 fusion proteins on a

Collagen-II antigen-coated sensor chip.

FIG. 6: Quality control analysis of the different IGF1-C11 variants (A) by Coomassie Blue staining of SDS-PAGE and (B) Size Exclusion Chromatography before and after concentration by ultrafiltration.

FIG. 7: Analysis of the different IGF1-C11 protein variants before (TO) and after 4 cycles of freeze/thawing (F/T). (A) SDS-PAGE under reducing conditions. Expected bands: IGF1-C11-HC=ca 57 Kda, Cll-LC=ca 23.4 KDa. (B) Western Blotting under reducing conditions using an anti IGF1 antibody for detection. Expected band: IGF1-C11-HC=ca 57 Kda and (C) Size Exclusion Chromatography using a Superdex 200 increase 5/150 GL column (GE Healthcare).

FIG. 8: Western Blot analysis of the IGF1-C11 protein variants after incubation in human serum for 24 or 120 h at 37° C. Samples were run under reducing conditions and visualized using an anti IGF1 antibody. Expected band: IGF1-C11-HC=ca 57 KDa.

FIG. 9: Cartoon of an exemplary embodiment of the present invention, comprising an IgG1 shaped antibody with heavy and light chain, and a peptide linker fused to the N-terminus of the heavy chain, with a growth factor fused to the N-terminus of the peptide linker. The peptide linker is shown, exemplarily, as the AKKAS linker, and the growth factor is shown, exemplarily, as IGF-1.

FIG. 10: Schematic representation of the IGF1-C11 mammalian expression vectors used for the production of the different IGF1-C11 formats in CHO-S cells. (A) IGF1-C11 in IgG format, (B) IGF1-C11 in scFv-Fc format and (C) IGF1-C11 in SIP format. SP=signal peptide, C11-LC=light chain sequence of the C11 antibody, pA=polyA signal; C11-VL=variable domain of the light chain of the C11 antibody, C11-VH=variable domain of the heavy chain of the C11 antibody, C11-Fc=C11 heavy chain fragment including the CH1 and Fc portions of the antibody, CH4=heavy chain constant region 4 of human IgE secretory isoform.

FIG. 11: Quality control analysis by SEC and SDS-PAGE of the IGF1-C11 fusion proteins (A) in IgG format and (B) in scFv-Fc format.

FIG. 12A-12D show the cloning strategy for IGF1 at the C or at the N terminus of an IgG or diabody.

FIG. 13A-13B show that while IGF1 is well-behaved when fused at the N terminus of both an IgG and a diabody, the fusion at the C terminus of a diabody results with a covalent dimer which would hamper its use in-vivo.

FIG. 14A-B show that while IGF1 retains its biological activity when fused at the N terminus of an IgG it does not induce proliferation of cells expressing IGF1 receptor when fused at the C terminus of an IgG. Unconjugated IGF1 is used as positive control and unstimulated cells as negative control.

FIG. 14C-D show that IGF1 retains its biological activity when fused at the N terminus of C11 antibody both in IgG and in diabody format. Unconjugated IGF1 is used as positive control and unstimulated cells as negative control.

FIG. 15 shows that different dosages of IGF1-C11 in a rat model of osteoarthritis promote cartilage regeneration following intrasynovial injection. Such improvement is statistically significant superior as compared to PBS, to IGF1 fused to the irrelevant KSF antibody, to IGF1 fused to the anti-EDA “F8” antibody and to FGF18, a growth factor which is the benchmark biologic for the treatment of OA.

FIG. 16 shows the fusion protein IGF1-C11 exhibits a persistent localization (>5 weeks after treatment) to cartilage following intrasynovial injection in a rat model of osteoarthritis. No staining is visible in the cartilages treated with PBS, IGF1-KSF and IGF1-F8.

FIG. 17 shows the staining of sheep cruciate ligaments and patellar tendons using C11 antibodies in IgG format (A) or using C11 antibody fluorescently labeled with IRDye800CW (B).

FIG. 18 shows the blood level of IGF1-C11, IGF1-F9 and IGF1-KSF after the first and second intra articular injections once a week of 600 μg/dose of IGF1-C11, IGF1-F9 and IGF1-KSF in a rat medial meniscus tear (MMT) model of osteoarthritis (OA). (A) Comparison of the blood level of IGF1-C11 and IGF1-KSF: IGF1-C11 shows the lowest blood exposure (B) Comparison of the blood level of IGF1-F9 and IGF1-KSF: IGF1-F9 shows a blood exposure which is similar to IGF1-KSF and higher than IGF1-C11.

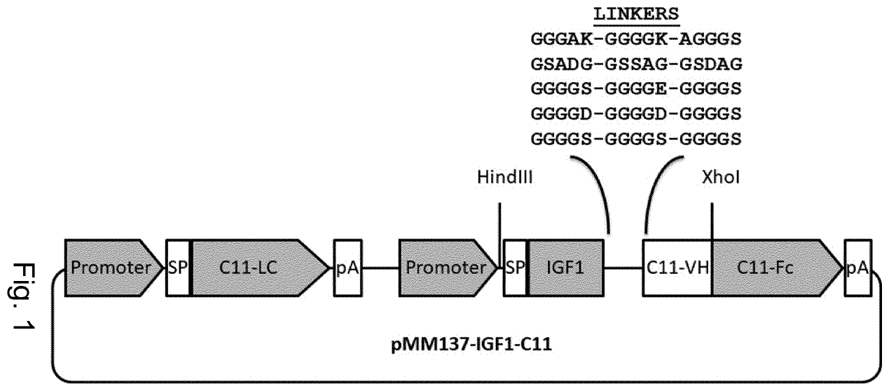

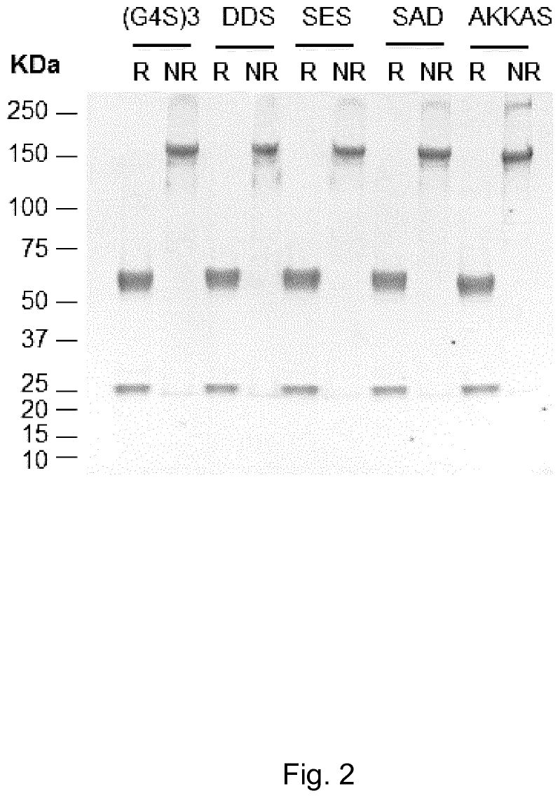

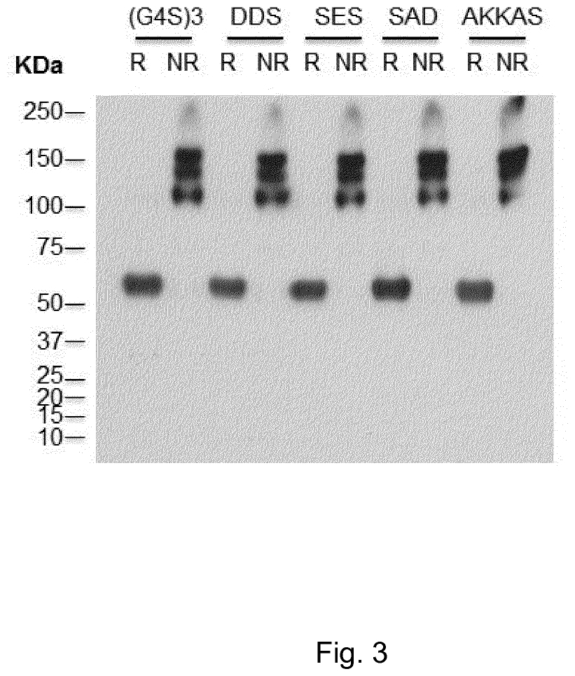

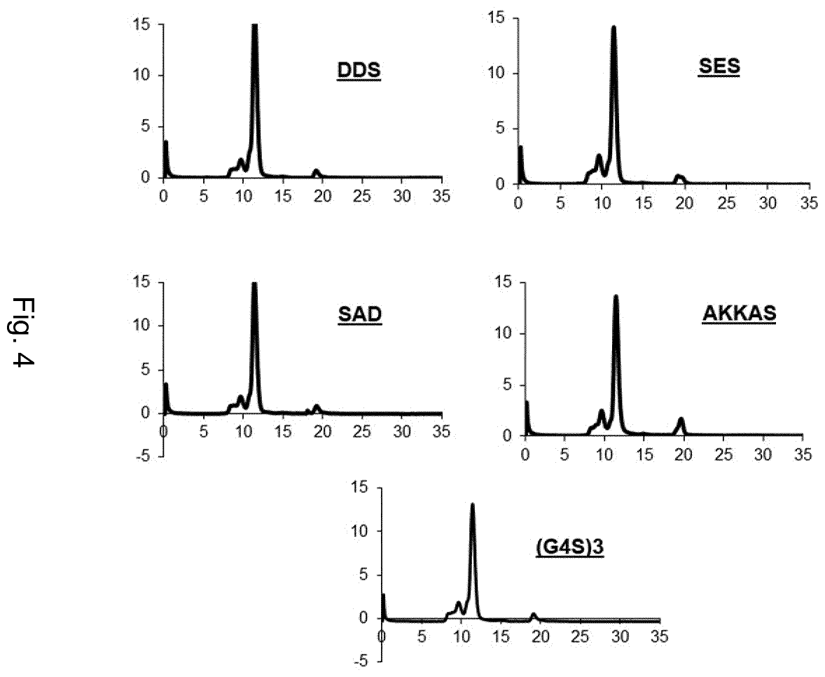

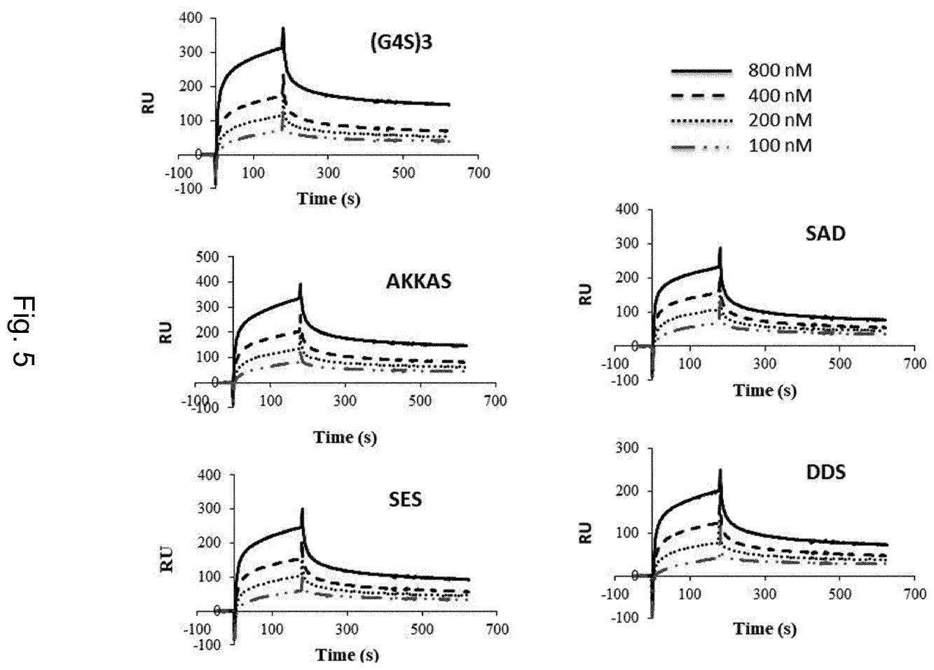

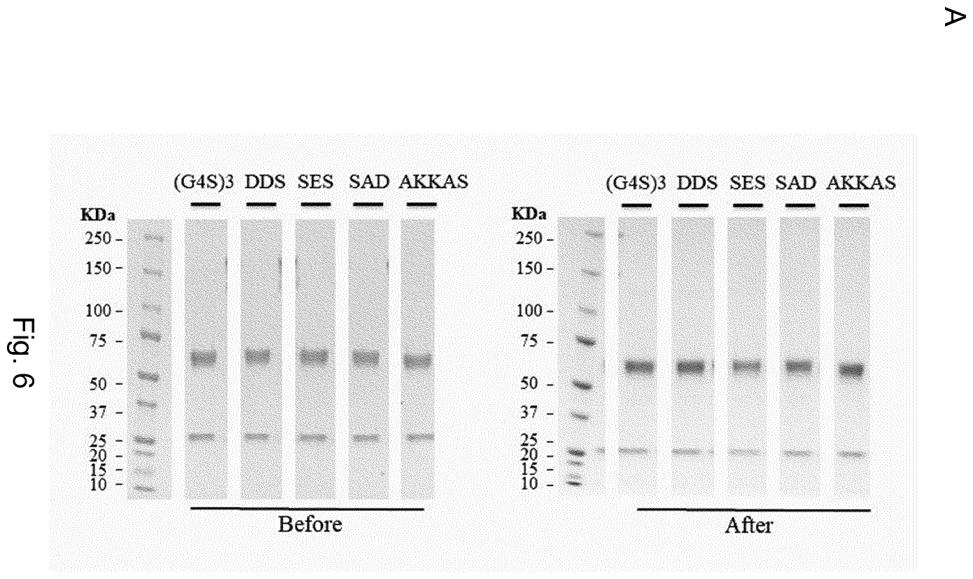

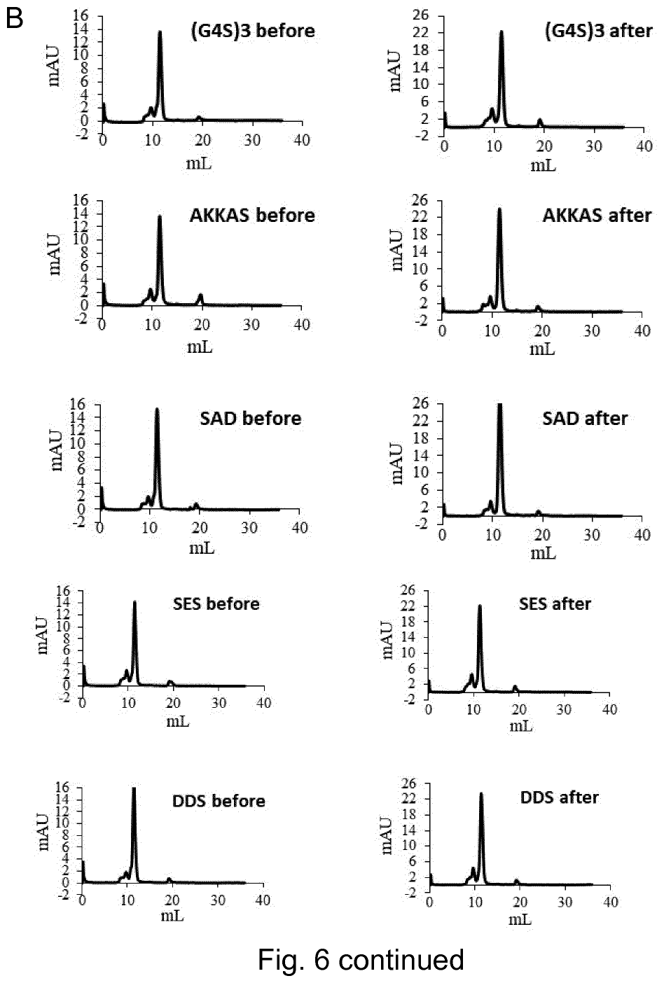

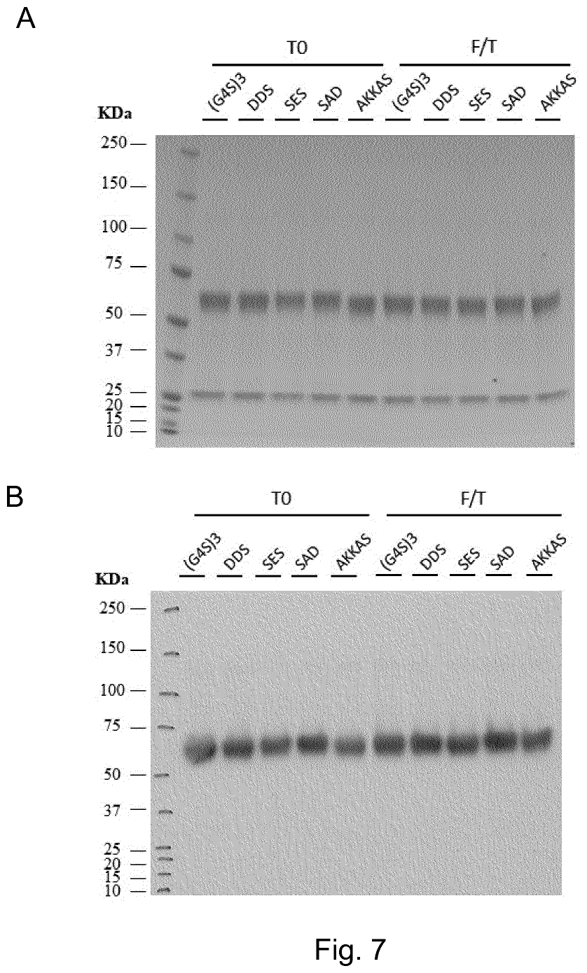

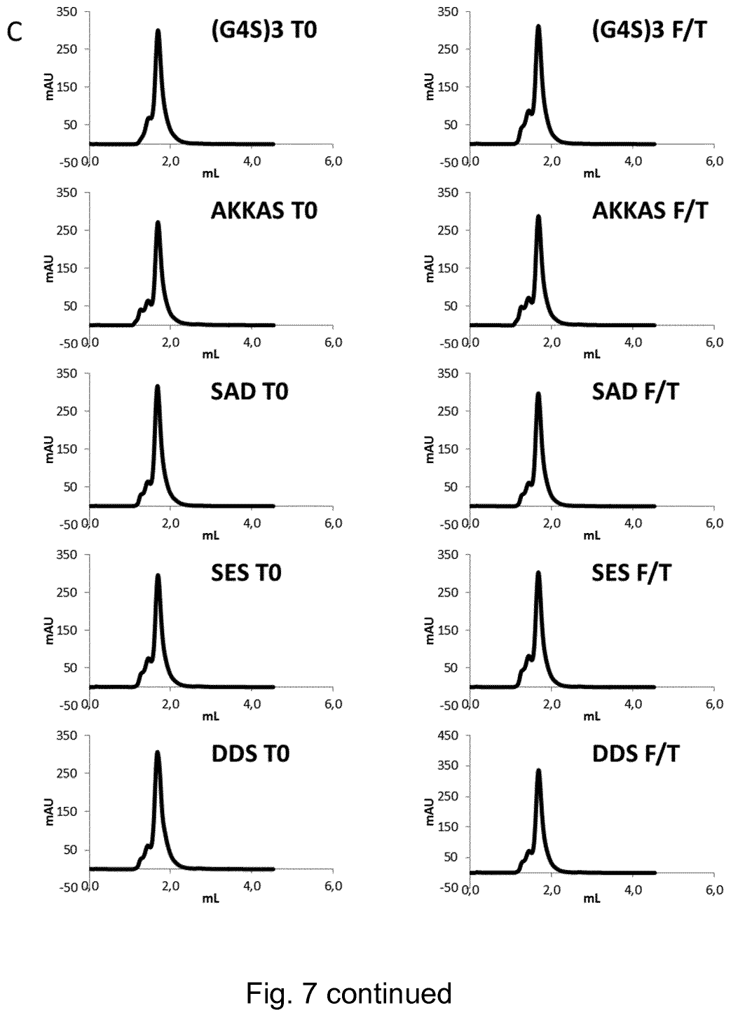

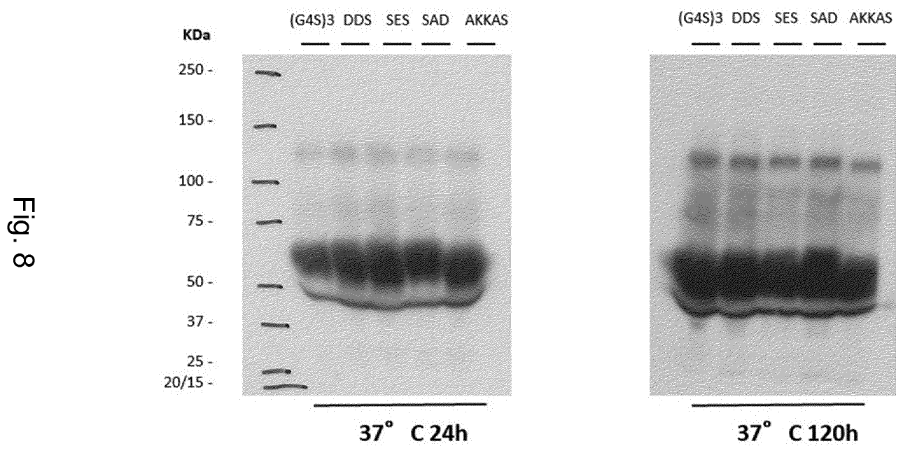

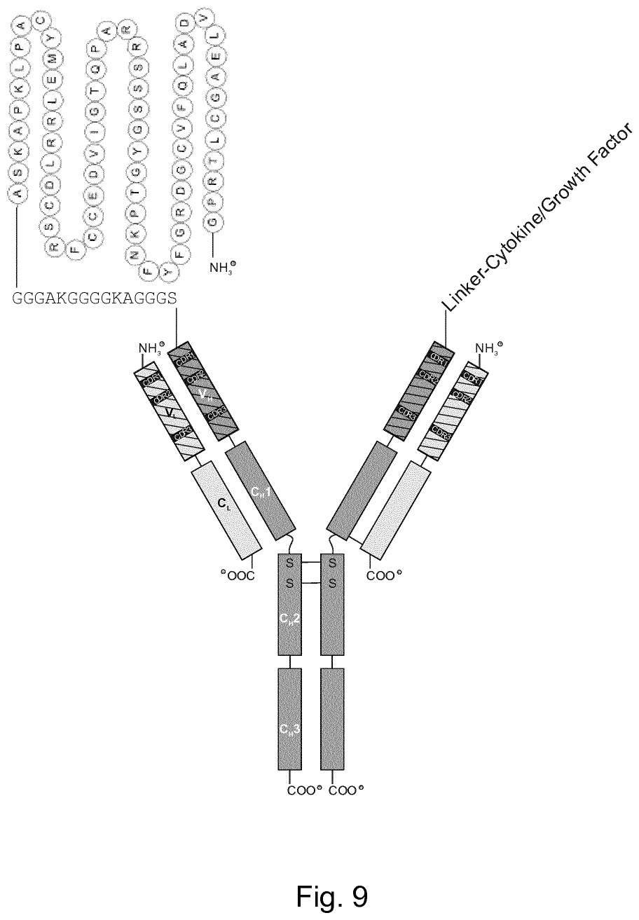

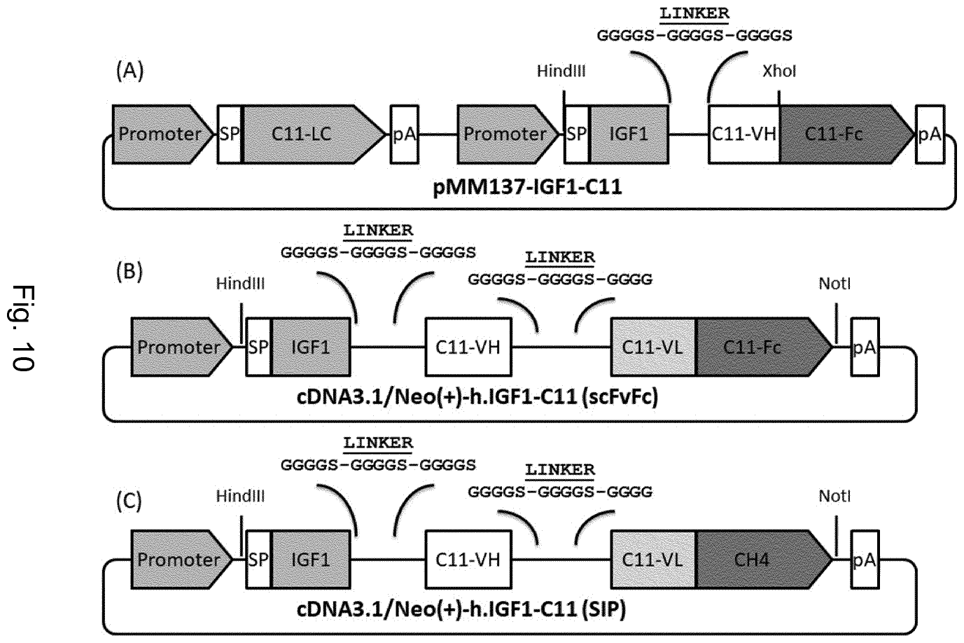

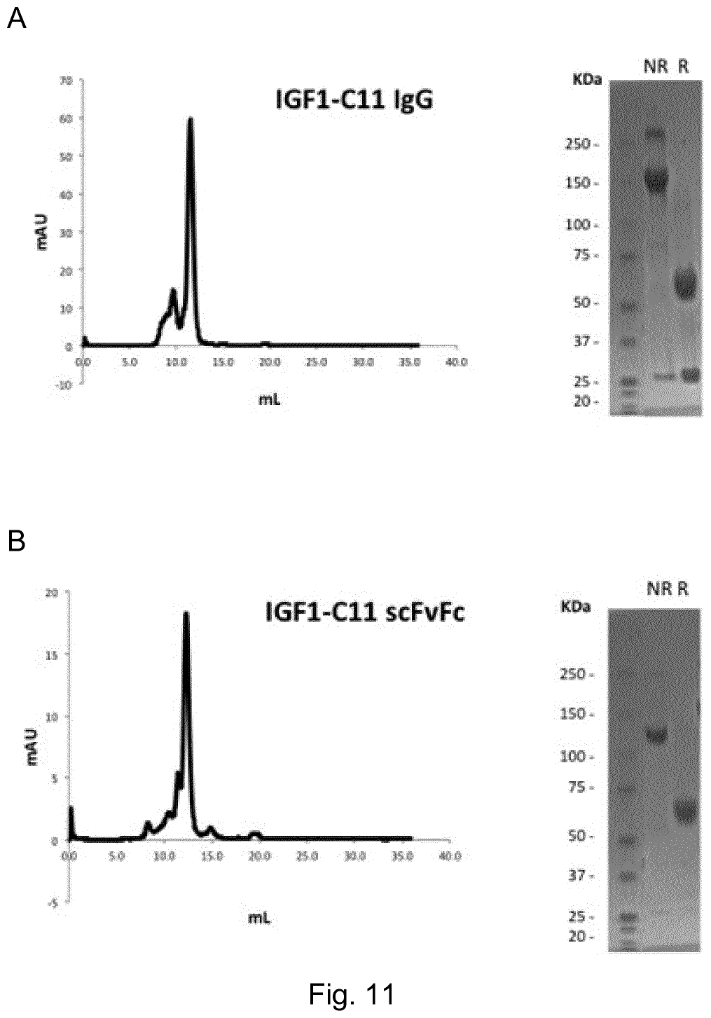

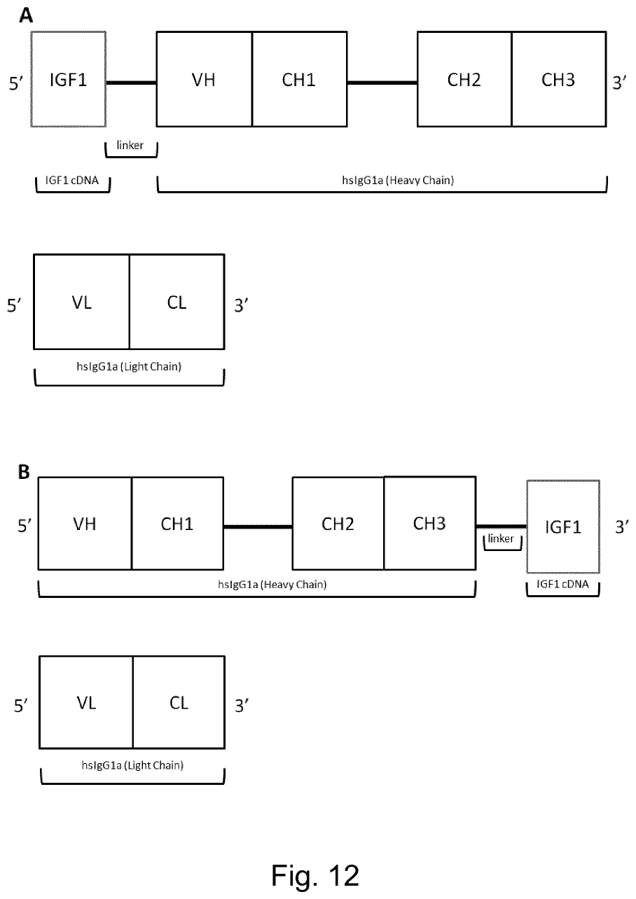

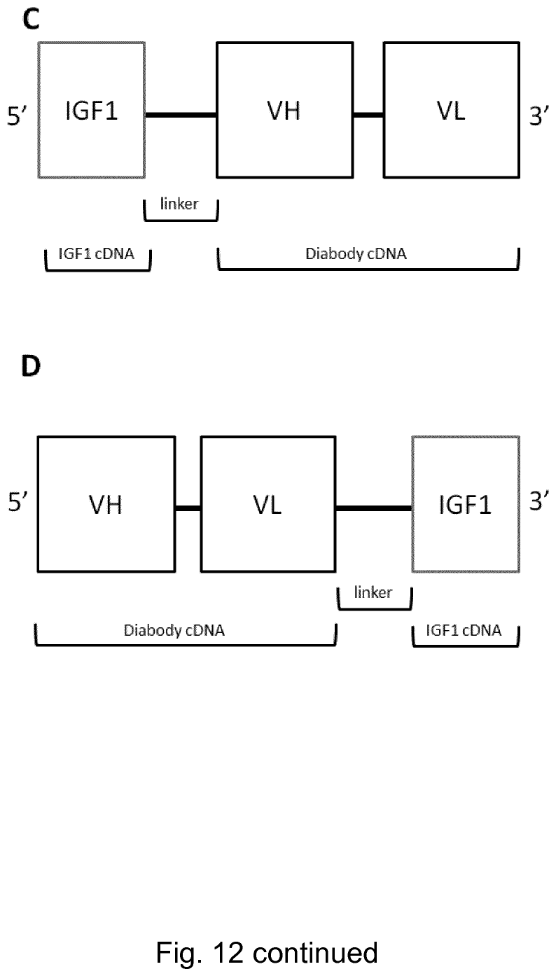

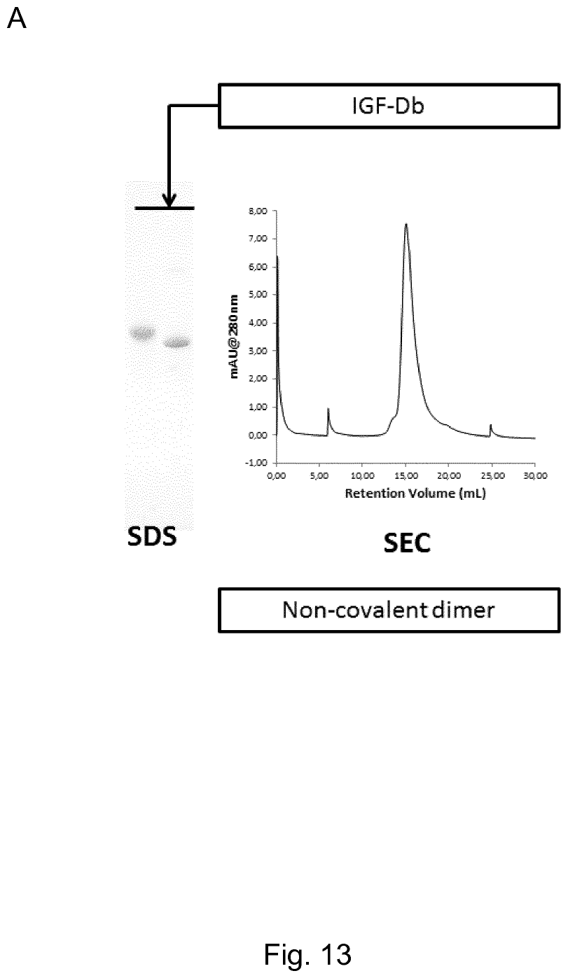

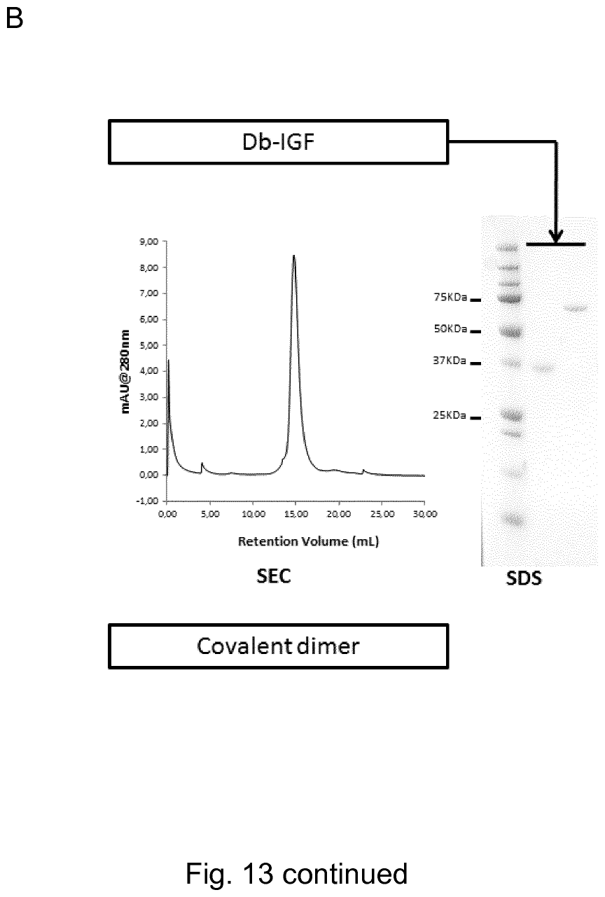

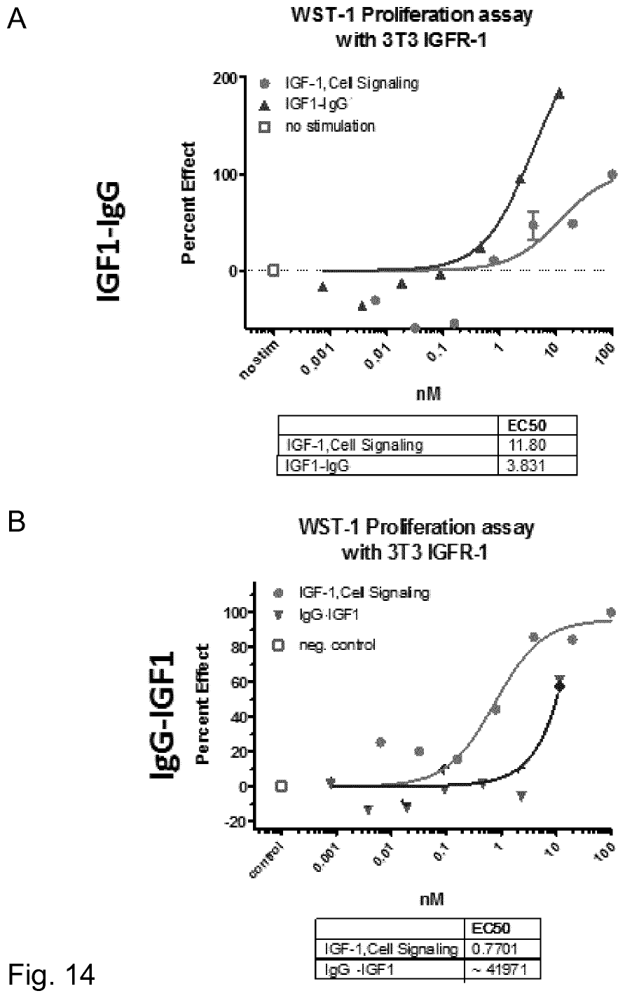

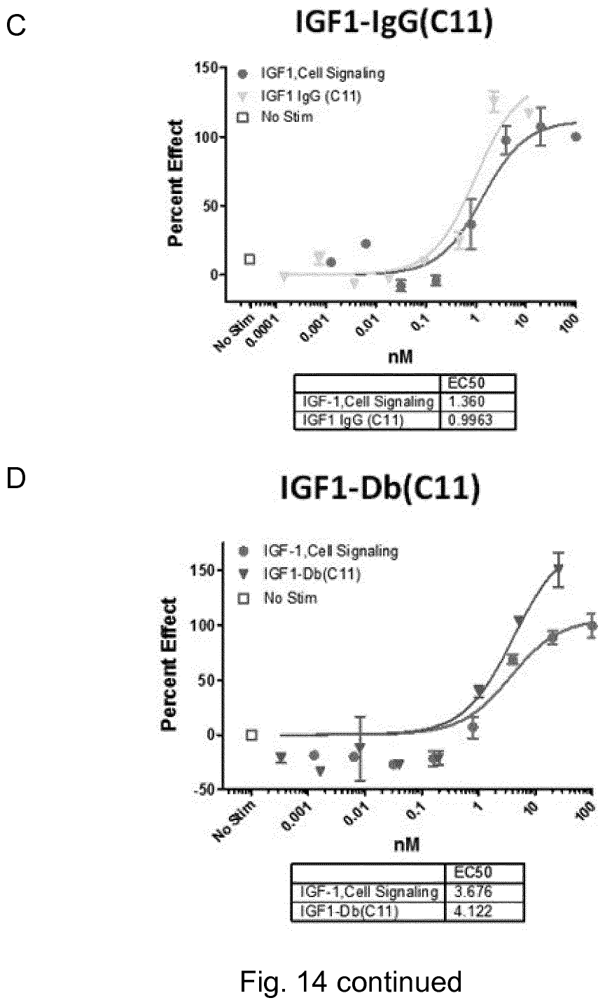

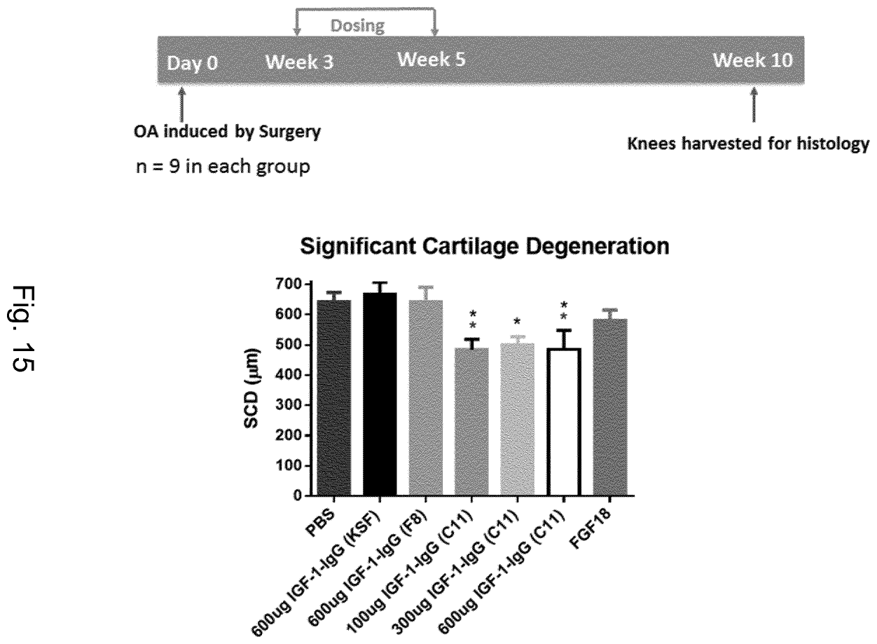

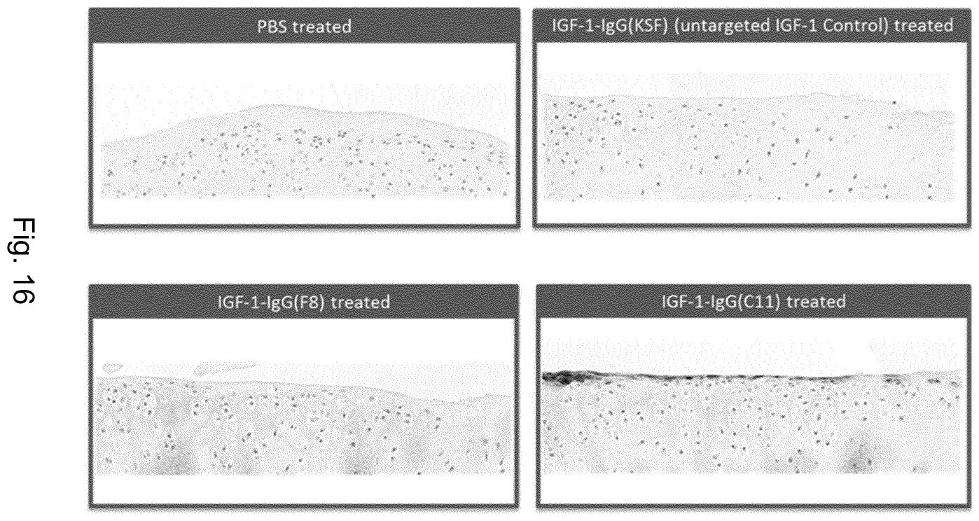

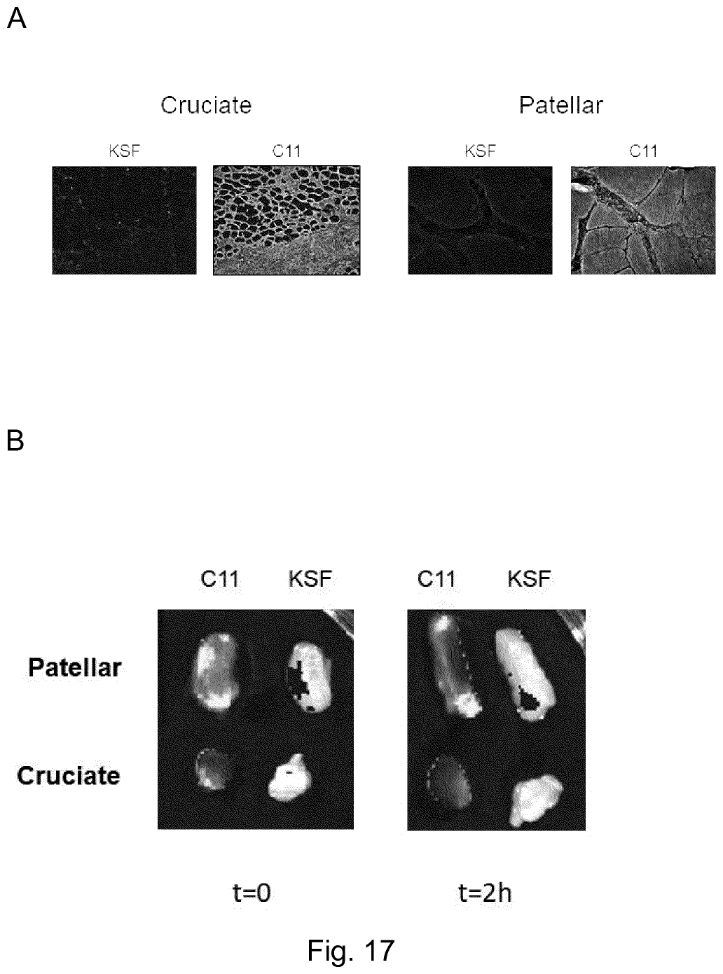

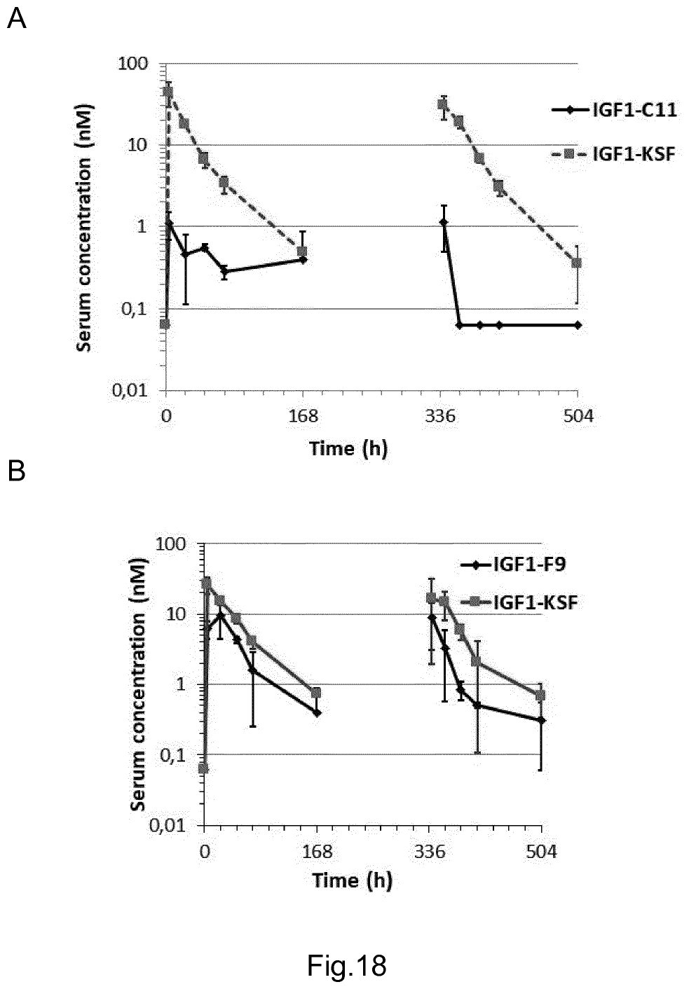

### REFERENCES

Aigner T. et al. (1992), Virchows Archives B Cell Pathol Incl Mol Pathol, 62, 337-345

Cheah K. S. et al. (1985), Biochem J, 229, 287-303

Chen et al. (2013), Adv Drug Deliv Rev, 65(10), 1357-1369

Eyre D. (2002), Arthritis Res, 4, 30-35

Gouttenoire J. et al. (2004), Biorheology, 41, 535-542

Köhler G and Milstein C (1975), Nature, 256, 495-497

Lee B. et al. (1989), Science, 244, 978-980

Strom C M and Upholt W. B. (1984), Nuc Acid Res, 12, 1025-1038

Lyman et al. (2009) J Bone Joint Surg Am ,91, 2321-2328

Freedman et al., (2003) Am J Sport Med, 31, 2-11

Brown & Carson Clin Sports Med., (1999) 18, 109-171

Rinderknecht and Humbel, Proc Natl Acad Sci U S A. 1976 July;73(7):2365-9.

Lee B. et al. (1989), Science, 244, 978-980

Kabat, E. A. et al., In: Sequences of Proteins of Immunological Interest, NIH Publication, 91-3242 (1991)

Chothia, C. and Lesk, A. M., J. Mol. Biol., 196, 901-917 (1987)

### SEQUENCES

| | | |
|---|---|---|
| 1 | C11 VL-CDR1 | RASQSVSSSYLA |
| 2 | C11 VL-CDR2 | GASSRAT |
| 3 | C11 VL-CDR3 | QQAIGFPQT |
| 4 | C11 VH-CDR1 | GFTFSSYAMS |
| 5 | C11 VH-CDR2 | AISGSGGSTYYADSVKG |
| 6 | C11 VH-CDR3 | TLAAFDY |
| 7 | C11-VL | EIVLTQSPGTLSLSPGERATLSCRASQSVSSSYLAWYQQKPGQAPRLLIYGASSR |
| | | ATGIPDRFSGSGSGTDFTLTISRLEPEDFAVYYCQQAIGFPQTFGQGTKVEIK |
| 8 | C11-VH | EVQLLESGGGLVQPGGSLRLSCAASGFTFSSYAMSWVRQAPGKGLEQVSAISGSG |
| | | GSTYYADSVKGRFTISRDNSKNTLYLQMNSLRAEDTAVYYCAKTLAAFDYWGQGT |
| | | LVTVSS |
| 9 | IGF-1 | GPETLCGAELVDALQFVCGDRGFYFNKPTGYGSSSRRAPQTGIVDECCFRSCDLR |
| | | RLEMYCAPLKPAKSA |
| 10 | AKKAS linker | GGGAKGGGGKAGGGS |
| 11 | DDS linker | GGGGDGGGGDGGGGS |
| 12 | SAD linker | GSADGGSSAGGSDAG |
| 13 | SES linker | GGGGSGGGGEGGGGS |
| 14 | (G4S)3 | GGGGSGGGGSGGGGS |
| 15 | IGF-1-C11-VH- | GPETLCGAELVDALQFVCGDRGFYFNKPTGYGSSSRRAPQTGIVDECCFRSCDLR |
| | AKKAS-variant | RLEMYCAPLKPAKSAGGGAKGGGGKAGGGSEVQLLESGGGLVQPGGSLRLSCAAS |
| | | GFTFSSYAMSWVRQAPGKGLEQVSAISGSGGSTYYADSVKGRFTISRDNSKNTLY |
| | | LQMNSLRAEDTAVYYCAKTLAAFDYWGQGTLVTVSS |
| 16 | IGF-1-C11-VH- | GPETLCGAELVDALQFVCGDRGFYFNKPTGYGSSSRRAPQTGIVDECCFRSCDLR |
| | DDS-variant | RLEMYCAPLKPAKSAGGGGDGGGGDGGGGSEVQLLESGGGLVQPGGSLRLSCAAS |
| | | GFTFSSYAMSWVRQAPGKGLEQVSAISGSGGSTYYADSVKGRFTISRDNSKNTLY |
| | | LQMNSLRAEDTAVYYCAKTLAAFDYWGQGTLVTVSS |
| 17 | IGF-1-C11-VH- | GPETLCGAELVDALQFVCGDRGFYFNKPTGYGSSSRRAPQTGIVDECCFRSCDLR |
| | SAD-variant | RLEMYCAPLKPAKSAGSADGGSSAGGSDAGEVQLLESGGGLVQPGGSLRLSCAAS |
| | | GFTFSSYAMSWVRQAPGKGLEQVSAISGSGGSTYYADSVKGRFTISRDNSKNTLY |
| | | LQMNSLRAEDTAVYYCAKTLAAFDYWGQGTLVTVSS |
| 18 | IGF-1-C11-VH- | GPETLCGAELVDALQFVCGDRGFYFNKPTGYGSSSRRAPQTGIVDECCFRSCDLR |
| | SES-variant | RLEMYCAPLKPAKSAGGGGSGGGGEGGGGSEVQLLESGGGLVQPGGSLRLSCAAS |
| | | GFTFSSYAMSWVRQAPGKGLEQVSAISGSGGSTYYADSVKGRFTISRDNSKNTLY |
| | | LQMNSLRAEDTAVYYCAKTLAAFDYWGQGTLVTVSS |
| 19 | IGF-1-C11-VH- | GPETLCGAELVDALQFVCGDRGFYFNKPTGYGSSSRRAPQTGIVDECCFRSCDLR |
| | (G4S)3-variant | RLEMYCAPLKPAKSAGGGGSGGGGSGGGGSEVQLLESGGGLVQPGGSLRLSCAAS |
| | | GFTFSSYAMSWVRQAPGKGLEQVSAISGSGGSTYYADSVKGRFTISRDNSKNTLY |
| | | LQMNSLRAEDTAVYYCAKTLAAFDYWGQGTLVTVSS |
| 20 | IGF1-C11 (scFv- | GPETLCGAELVDALQFVCGDRGFYFNKPTGYGSSSRRAPQTGIVDECCFRSCDLR |
| | Fc)-(G4S)3- | RLEMYCAPLKPAKSAGGGGSGGGGSGGGGSEVQLLESGGGLVQPGGSLRLSCAAS |
| | variant | GFTFSSYAMSWVRQAPGKGLEQVSAISGSGGSTYYADSVKGRFTISRDNSKNTLY |
| | | LQMNSLRAEDTAVYYCAKTLAAFDYWGQGTLVTVSSGGGGSGGGGSGGGGEIVLT |
| | | QSPGTLSLSPGERATLSCRASQSVSSSYLAWYQQKPGQAPRLLIYGASSRATGIP |
| | | DRFSGSGSGTDFTLTISRLEPEDFAVYYCQQAIGFPQTFGQGTKVEIKEPKSCDK |
| | | THTCPPCPAPELLGGPSVFLEPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEWESNGQP |
| | | ENNYKTTPPVLDSDGSFELYSKLTVDKSRWQQGNVESCSVMHEALHNHYTQKSLS |
| | | LSPGK |
| 21 | IGF1-C11 (SIP)- | GPETLCGAELVDALQFVCGDRGEYENKPTGYGSSSRRAPQTGIVDECCFRSCDLR |
| | (G4S)3-variant | RLEMYCAPLKPAKSAGGGGSGGGGSGGGGSEVQLLESGGGLVQPGGSLRLSCAAS |
| | | GFTFSSYAMSWVRQAPGKGLEQVSAISGSGGSTYYADSVKGRETISRDNSKNTLY |
| | | LQMNSLRAEDTAVYYCAKTLAAFDYWGQGTLVTVSSGGGGSGGGGSGGGGEIVLT |
| | | QSPGTLSLSPGERATLSCRASQSVSSSYLAWYQQKPGQAPRLLIYGASSRATGIP |
| | | DRFSGSGSGTDFTLTISRLEPEDFAVYYCQQAIGFPQTFGQGTKVEIKSGGSGGP |
| | | RAAPEVYAFATPEWPGSRDKRTLACLIQNFMPEDISVQWLHNEVQLPDARHSTTQ |
| | | PRKTKGSGEFVFSRLEVTRAEWEQKDEFICRAVHEAASPSQTVQRAVSVNPESSR |
| | | RGGC |

1. Fusion protein comprising
an antibody, or a fragment or derivative thereof retaining target binding properties, and an insulin-like growth factor 1 (IGF-1),
wherein said antibody, or fragment or derivative thereof, and said growth factor are linked by a peptide linker,
wherein the peptide linker is fused to the N-terminus of at least one peptide chain of the antibody, or the fragment or derivative thereof.

2. Fusion protein according to claim 1, wherein said antibody, or fragment or derivative thereof, is specifically binding to a collagen.

3. Fusion protein according to claim 1, wherein said antibody, or fragment or derivative thereof, is capable of binding both collagen I and collagen II.

4. (canceled)

5. The fusion protein according to claim 1, wherein said antibody is a monoclonal antibody selected from any of the group consisting of hybridoma-derived, chimerized, humanized or human antibody.

6. The fusion protein according to claim 1, wherein said antibody is a monoclonal antibody selected from any of the group consisting of canine or caninized antibody, and/or equine or equinized antibody.

7. The fusion protein according to claim 1, wherein the antibody is in at least one format selected from the group consisting of
IgG and diabody.

8. The fusion protein according to claim 1,
wherein the linker comprises an amino acid sequence selected from any of the group consisting of
a)
(SEQ ID NO: 10)
GGGAKGGGGKAGGGS
b)
(SEQ ID NO: 11)
GGGGDGGGGDGGGGS
c)
(SEQ ID NO: 12)
GSADGGSSAGGSDAG
d)
(SEQ ID NO: 13)
GGGGSGGGGEGGGGS,
and
e)
(SEQ ID NO: 14)
GGGGSGGGGSGGGGS.

9. The fusion protein according to claim 1, wherein said antibody has the following CDRs

| | |
|---|---|
| VL-CDR1 | SEQ ID NO: 1 |
| VL-CDR2 | SEQ ID NO: 2 |
| VL-CDR3 | SEQ ID NO: 3 |
| VH-CDR1 | SEQ ID NO: 4 |
| VH-CDR2 | SEQ ID NO: 5 |
| VH-CDR3 | SEQ ID NO: 6. |

10. The fusion protein according to claim 1, wherein said antibody has light chain variable domain (VL) domain according to SEQ ID NO: 7 and a heavy chain variable domain (VH) domain according to SEQ ID NO: 8.

11. (canceled)

12. The fusion protein according to claim 1, wherein said fusion protein is a recombinant fusion protein.

13. The fusion protein according to claim 1, which fusion protein comprises at least one chain comprising the amino acid sequence of SEQ ID NO: 15.

14. The fusion protein according to claim 1, which fusion protein comprises at least one chain comprising the amino acid sequence of SEQ ID NO: 16.

15. The fusion protein according to claim 1, which fusion protein comprises at least one chain comprising the amino acid sequence of SEQ ID NO: 17.

16. The fusion protein according to claim 1, which fusion protein comprises at least one chain comprising the amino acid sequence of SEQ ID NO: 18.

17. The fusion protein according to claim 1, which fusion protein comprises at least one chain comprising the amino acid sequence of SEQ ID NO: 19.

18. The fusion protein according to claim 1, which fusion protein comprises at least one chain comprising the amino acid sequence of SEQ ID NO: 20.

19-23. (canceled)

24. A method of treating a human or animal subject which is suffering from, at risk of developing, or diagnosed for
(a) arthritis, preferentially osteoarthritis, or
(b) ligament or tendon injury, which method comprises administering to said subject a fusion protein according to claim 1.

25. (canceled)

26. The method according to claim 24, wherein the fusion protein is administered by an intra-articular injection.

27. An ex vivo method of pre-treatment of ligaments or tendons used for reparative surgery, which method comprises incubating a ligament or tendon with a fusion protein according to claim 1.
